(19) United States
(12) Patent Application Publication
(10) Pub. No.: US 2013/0150686 A1
(43) Pub. Date: Jun. 13, 2013

(54) Human Care Sentry System

(71) Applicants: Donald Clark Fronterhouse, Albuquerque, NM (US); Thomas Umberson Mead, Sandia Park, NM (US); Patrick Sean McGuirk, Albuquerque, NM (US); Kenneth Brian Center, Lakewood, CO (US)

(72) Inventors: Donald Clark Fronterhouse, Albuquerque, NM (US); Thomas Umberson Mead, Sandia Park, NM (US); Patrick Sean McGuirk, Albuquerque, NM (US); Kenneth Brian Center, Lakewood, CO (US)

(73) Assignee: PnP INNOVATIONS, INC, Albuquerque, NM (US)

(21) Appl. No.: 13/313,755

(22) Filed: Dec. 7, 2011

(51) Int. Cl. A61B 5/0205; A61B 5/145; A61B 5/021; G01G 19/44; A61B 5/11; G06F 15/16; H04N 7/15; A61B 5/1455; A61B 5/024

(52) U.S. Cl. 600/323; 600/322; 600/365; 600/485; 600/508; 600/483; 600/595; 709/201; 348/14.04; 702/173; 348/E07.083

(57) ABSTRACT

Disclosed is a process and composition of matter to support safe, assisted, independent living. The process is to create ubiquitous monitoring of the invention user's activities, physiology, and environment; analyze information from monitoring and sensing devices; and act on the information in a prioritized manner to address emergent events, and potentially undesirable conditions. The invention uses a software architecture and schema called Adaptive Scalable Plug&play Infrastructure for Responsive Engineering (ASPIRE).

### BACKGROUND OF THE INVENTION

1. Field of the Invention

The invention relates to the use of an advanced software architecture and schema that facilitate the creation of expandable, scalable networks of hardware and software devices to support safe, independent, more productive and fulfilled living.

2. Description of Prior Art

This section will discuss representative examples of prior art that are germane to this invention. Because the invention uses an improved ‘plug and play’ software approach to create a system of components working together to create capabilities, the discussion will, in order, address monitoring and intervention systems, data movement within systems, monitoring devices, data and information processing, and then software architecture and schema. At the end of this section is a summary of the current state of the art.

### Monitoring Systems:

U.S. Pat. No. 7,991,625 describes a system for providing expert care to a basic care medical facility (BCMF) from a remote location. The system facilitates real-time, continuous assessment of patients receiving care in a BCMF that is not generally equipped to provide expert medical care on a twenty-four basis. Patient monitoring equipment acquires monitored data elements from a patient monitoring station and transmits the monitoring data over a network to a remote command center. The remote command center also receives other patient data to the extent available from the BCMF. Alternatively, the patient monitored data is sent to a remote command center along with patient data at a pre-established time or when requested by remote command center. The delivery of stored monitoring data and patient data may be expedited if an urgent consultation is warranted. A rules engine continuously applies a patient-specific rule or rule set to the data elements selected from the assessment data from each BCMF monitored patient to determine whether intervention is warranted. Patient specific rules may be created that are consistent with the capabilities of the BCMF.

This invention is focused on supporting a patient who has existing problems that warrant entering a special facility, the BCMF. It does not support the continuous monitoring of individuals in routine living activities in their homes or work environments, nor monitoring of the environment and facilities in the home. This invention does not describe a back up capability to the remote command center. This decreases the robustness of the system. This invention does not support the ability of ancillary persons (such as family, friends, or routine care givers) being able to connect by video conference to a patient to provide the kind of social interaction and support that maintains good mental health. This invention uses a rules engine for several of its key functions such as assessing data and identifying conditions warranting management by a health care provider, and when to intervene or what actions to take. The invention does not talk about how the actual rules are implemented into applications and how those applications and the hardware associated with the invention are hosted by computers. Under current art, there is no hardware or software standard that easily enables the joining of hardware or software (such as data analysis or inference algorithms) to systems. As a result, a software writer must write software that is potentially unique or proprietary for each system arrangement, that is, he must create specific customization code for each configuration. This hampers the ability of this invention to rapidly integrate new capabilities that become available.

U.S. Pat. No. 7,970,620 describes an invention comprising a networked system for identifying an individual, communicating information to the individual, and remotely monitoring the individual. The system includes a remotely programmable apparatus that occasionally connects to a server via a communication network such as the internet. The remotely programmable apparatus interacts with the individual in accordance with a script program received from the server. Among other capabilities, the script program may instruct the remotely programmable apparatus to identify the individual, to communicate information to the individual, to communicate queries to the individual, to receive responses to the queries, and to transmit information identifying the individual and the responses from the remotely programmable apparatus to the server. Information identifying the individual may be obtained via a biometrics sensor, a data card, a remote monitoring device, or the interception of data from a separate information system. The information identifying the individual may be used by either or both the server system and remotely programmable apparatus for security, customization and other purposes. As the present invention has multi-user capabilities, it can be used in a public place, such as a pharmacy or health care clinic. The multi-user capabilities also allow collection and tracking of user data for the healthcare industry.

This invention acts on data taken periodically and collected for action on a central server. It does not perform continuous monitoring and enable reaction to emergent events. Also, it does not monitor the environment. Furthermore, the invention does not use video teleconferencing to communicate with the patient when there is a caretaker or medical professional interacting with the patient. As a result, the system does not have the ability to let caretakers or medical professionals see or hear the patient and detect mood, expressions, or even skin color.

U.S. Pat. No. 7,937,461 describes a system for monitoring a user in a user living area. The system includes a system controller and an activity detection subsystem. The activity detection subsystem monitors a daily living activity of the user and provides information representative of the daily living activity to the system controller. The system controller includes a control circuit which generates a control signal in response to the daily living activity information obtained by the activity detection subsystem. Control information from the system controller is applied by way of a control information communication channel both to the activity detection subsystem and to a remote monitoring site. The activity detection subsystem may be a system for determining the movement of the user around the home, medication compliance by the user, problems with usage of stoves or other potentially dangerous appliances, and selected auxiliary appliances.

This invention has a good capability to address emergent events. It includes use of interactive television as one means of a user communicating with a remote monitoring center. The ability to see visual cues of a patient as well as potentially seeing injuries aids in a care giver's ability to make correct assessments of the patient's condition and needs. This invention does not provide for the ability of an attendant at a remote monitoring center being able to forward a video call to a third party who would also benefit from seeing and hearing a patient. Additionally, the invention does not include the ability of a patient to use his video capability to directly call or be called by care giver, medical professionals, family, and friends. This is an important capability to provide mental health support and keep a patient connected to society and loved ones.

This invention also does not provide for a back-up plan for having access to a remote monitoring center as well as access to patient data that may be needed to address an emergent need.

This invention describes the system controller, and for that matter, all computers in the invention, as being programmed to perform controlling a wide range of monitoring devices, performing data analysis, and controlling a wide range of appliances and devices. The invention does not talk about how the actual software is implemented into applications and how those applications and the hardware associated with the invention are hosted by computers. Under current art, there is no hardware or software standard that easily enables the joining of hardware or software (such as data analysis or inference algorithms) to systems. To actually create such a system requires the use of significant numbers of software modules that connect the various sensors and computers into a network as well as apply logic to understand what is happening in the environment and with the user. The patent does not prescribe a software architecture or schema. As a result, under current software and hardware art, there is a high probability that a user of the invention must write specific software that translates information for every hardware or software element to the format that can be understood the corresponding hardware or software element using that information. Similarly, there is high risk that the formatting or describing of the transport information will also require specific software to enable information to flow between elements of hardware and software. The impact of this is that the system may be extremely expensive to build with a wide variety of hardware sensors and software modules that collect data, create information, or control devices in the system. As a result, a software writer must write software that is potentially unique or proprietary for each system arrangement, i.e., he must create specific customization code for each configuration. Furthermore, this software approach hampers the ability of this invention to rapidly integrate new capabilities that become available.

U.S. Pat. No. 6,364,834 describes an integrated medical monitoring system comprising at least one patient monitor, at least one central monitor, and at least one remote access device which are tied together through an integrated communications link is disclosed. The communications between various components of the system are bi-directional, thereby affording the opportunity to establish monitoring parameters from remote locations, provide interactive alarms and monitoring capabilities, and provide data exchange between components of the system.

This invention provides limited safety and well being oversight for a person in that it monitors a person's physiological status but does not monitor a person's environment such as his home. In communication with a person being monitored, the communication is limited to text and voice which misses the advantages of video interaction.

This invention also does not provide for a back-up plan for having access to a remote monitoring center as well as access to patient data that may be needed to address an emergent need.

This invention describes the functions that software will perform within various parts of the system. The invention does not talk about how the actual software is implemented into applications and how those applications and the hardware associated with the invention are hosted by computers. Under current art, there is no hardware or software standard that easily enables the joining of hardware or software (such as data analysis or inference algorithms) to systems. As a result, a software writer must write software that is potentially unique or proprietary for each system arrangement, i.e., he must create specific customization code for each configuration. This hampers the ability of this invention to rapidly integrate new capabilities that become available.

U.S. Patent Application Publication No. 2006/0149140 describes an apparatus, system, and method for determining a patient's condition. A system for determining a patient's condition, in one embodiment, comprises a computer processor device, an interface between the computer processor and one or more patient monitoring devices, and a software program configured to interpret patient input data, diagnose the patient's condition, and recommend treatment options. A method for determining a patient's condition may comprise receiving patient data from one or more patient monitoring devices, consulting a treatment database, determining a diagnosis, and converting a diagnosis into a treatment.

The system application is targeted towards a medical crisis type environment, such as an emergency room, where it will integrate inputs from various devices and output diagnosis and treatment information. The system does not propose a monitoring system for physiologic or emergent environmental conditions applicable to the home environment.

U.S. Pat. No. 4,838,275 describes an apparatus in each patient's home that includes special furniture on which the patient lies and sits, and embedded in which are devices that automatically sense multiple parameters related to the patient's health. The patient cooperates only passively. The parameters are so chosen--and are sufficiently numerous and accurate—as to provide in the aggregate a comprehensive profile of the patient's general state of health. The apparatus also generates electronic health-parameter signals related to the sensed parameters, and it transmits these signals from the patient's home to a central surveillance and control office. Equipment there receives the signals, displays corresponding indicia of the parameters, and transmits control signals back to the patient's apparatus. Two-way voice communication between the patient and a highly trained observer at the central office supplements the electronic measurements. The observer conducts routine diagnostic sessions except when an emergency is noted from these sessions or from a patient-initiated communication. The observer determines whether a non-routine therapeutic response is required, and if so facilitates such a response. Selection among emergency cases follows a highly refined emergency-priority hierarchy.

This patent does not provide for monitoring of emergent events in the environment not does it provide for continuous monitoring of the patient.

U.S. Pat. No. 5,544,649 describes an ambulatory (in the home) patient health monitoring system wherein the patient is monitored by a health care worker at a central station, while the patient is at a remote location. The patient may be a person having a specific medical condition monitored or may be an elderly person desiring general medical surveillance in the home environment. Cameras are provided at the patient's remote location and at the central station such that the patient and the health care worker are in interactive visual and audio communication. A communications network such as an interactive cable television is used for this purpose. Various medical condition sensing and monitoring equipment are placed in the patient's home, depending on the particular medical needs of the patient. The patient's medical condition is measured or sensed in the home and the resulting data is transmitted to the central station for analysis and display. The health care worker then is placed into interactive visual communication with the patient concerning the patient's general well being, as well as the patient's medical condition. Thus, the health care worker can make “home visits” electronically, twenty-four hours a day.

This system does not monitor a person or the person's environment for emergent conditions nor provide a way to address emergent issues. This system does all data processing at a central location—it does not attempt to perform analysis in the home. Furthermore, there is no description of a back-up capability for the central monitoring station which puts the patient at risk of losing support in the event of communications disruptions with the central station. The software architecture and schema do not support an easily scalable and expandable network of hardware and software components. The video communications system does not address the ability of family, caregivers, and friends to use the system to supplement support provided by the central monitoring station.

U.S. Pat. No. 6,611,206 describes an alarm system that monitors conditions of an independent person, yet one requiring some supervision, such as an elderly person living alone at home. The system monitors a variety of independent signals and combines them to recognize subtle cues that may indicate there will be a need for intervention by a supervisor.

U.S. Pat. No. 6,968,294 describes a monitoring system for an infant, child, invalid, or other person requiring care uses computer vision and hearing, and inputs of other modalities, to analyze the status of a caretaker and/or cared-for person and its environment. The conditions are classified as normal or alarm conditions and an informative alarm signal is generated which may include records of the vision audio and other inputs. The system also has the ability to solicit responses from the occupants to stimulate a classifiable input to reduce ambiguity in its state signal.

The above two inventions describe artificial intelligence capabilities running on computers to analyze data coming from various sensors in a home supporting a sick or elderly person. The invention does not talk about how the actual software is implemented into applications and how those applications and the hardware associated with the invention are hosted by computers. Under current art, there is no hardware or software standard that easily enables the joining of hardware or software (such as data analysis or inference algorithms) to systems. As a result, a software writer must write software that is potentially unique or proprietary for each system arrangement, i.e., he must create specific customization code for each configuration. This hampers the ability of this invention to rapidly integrate new capabilities that become available.

### Data Movement:

U.S. Pat. No. 8,040,238 describes a solution to backhauling health information. The present invention utilizes a mesh network to backhaul the health information. The system includes a plurality of first tags, a mesh network, and an information engine. Each of the tags represents a first object. The mesh network preferably includes a plurality of plug-in sensors located within the facility. At least one node in the mesh network operates as healthcare device. The information engine is in communication with the mesh network and determines a position location of the healthcare device and an operation of the healthcare device.

This solution to moving health information addresses networks over which to move data but does not address the effort needed to write software nor to manage the data messaging traffic flowing between sensors and the information engine. Each sensor must have software written to enable it to receive and understand information from tags and must have software written to put tag or its own native information in format that enables it to be used by an application in the information engine. Similarly, each application in the information engine must have software written to enable it to understand and use the data coming to it. The invention does not address how the network is managed. It acknowledges that data could be sent continuously, at preset time intervals, or dynamically. This has the potential of creating high traffic on the data transmission paths that could cause delays or loss of information to the applications running on the information engine. Additionally, the invention does not address how the information engine knows when sensors are added or dropped from the system. This can result in critical functions not being accomplished.

### Monitoring Devices:

U.S. Pat. No. 7,477,143 is an example of a monitoring device. The patent describes how home safety and security are provided using a bedside unit to monitor an audible personal security pendant and to send notification signals to the appropriate communication site. Improved reliability is achieved by comparing a stored signal signature to the real time digital pattern representing sounds received using digital acoustic signature recognition technology.

This invention addresses only acoustic monitoring of the environment, of a person, or signals coming from monitoring devices that are built or modified to emit audible signals. It describes the use of analysis software to interpret the data but it does not address a standardized software architecture or schema. For each acoustic signature analysis application running on the computer and potentially, for each piece of hardware that is part of the invention, software must be written to make the data from the hardware devices compatible with the specific algorithms. Similarly, the software algorithms running in the processor must all have software written tailoring them to be able to use data from each data source. As sources change, the algorithms may potentially require new tailoring software.

U.S. Pat. No. 7,324,824 disclosed a plug-in network appliance. In one aspect, a network appliance performs a bridge between two wireless communication formats. In another aspect, a network appliance is deployed to perform position location services. In another aspect, a mesh network comprising one or more network appliances is deployed. A mesh network comprising one or more network appliances may be deployed to perform position location services. A plug-in form factor is described. A network appliance may convert power received from a plug-in a first format to power in a second format for powering various components. A network appliance may connect with a wireless network and/or a network connected through a plug. A plug-in network appliance may connect to a weight-bearing outlet. Various other aspects are also presented.

The network appliance in this invention does not include features to make it have built-in safety capabilities such as a smoke or carbon monoxide detector, an emergency light, or a speaker to enable relayed verbal information nor a microphone to enable capture of verbal information from a person in the proximity of the network appliance.

U.S. Pat. No. 8,034,294 describes an invention to generally provide medical monitoring devices, and corresponding systems, that includes at least one sensor for detecting usage of at least one consumable, an electronic controller electrically connected to the sensor to receive a signal produced when consumable usage is detected, and an electronic memory electrically connected to the controller for storing consumable usage data thereon. The medical monitoring device may further include at least one sensor for determining one or more physiological parameter of a subject and a display device for displaying the physiological parameter. The medical monitoring device may also include a communications unit for communicating stored data, such as usage and physiological parameter data, over a communication network to a remote computer.

This invention does not monitor the patient or the environment for emergent events. This invention does not describe the actual software architecture nor applications that enable data from medical monitoring devices to be moved within the invention nor applications tracking consumables used in connection with medical monitoring devices, nor applications for making determinations relating to usage, resupply, record keeping, and payment. For each application running on a processor in the invention and for each piece of hardware that is part of the invention, software must be written to make the data from the hardware devices compatible with the specific algorithms. Similarly, the software algorithms running in the inference engine must all have software written tailoring them to be able to use data from each data source. As sources change, the algorithms may potentially require new tailoring software.

U.S. Pat. No. 7,961,109 describes a fall detecting apparatus and method and an emergency aid system and method using the same. The fall detecting apparatus includes a storing unit configured to store fall data vectors; an angular velocity measuring unit configured to measure an angular velocity value; an acceleration measuring unit configured to measure an acceleration value; an acceleration extracting unit configured to extract a kinetic acceleration value and a gravitational acceleration value by filtering the acceleration value measured by the acceleration measuring unit; and a fall determining unit configured to convert the angular velocity value, which is measured by the angular velocity measuring unit, and the kinetic acceleration value and the gravitational acceleration value, which are extracted by the acceleration extracting unit, into a fall data vector, and determine a use's fall by comparing the converted fall data vector with the fall data vector stored in the storing unit.

This invention relies on stored acceleration profiles to determine if a fall has occurred. It does not provide for fall corroboration by other means such as subsequent movement typical of walking, or sensing motion by motion detectors, or verbally interrogating the person if he needs help. This invention has the potential of false or nuisance detections that could cause a user to stop using it and become unprotected.

U.S. Pat. No. 7,502,498 describes a monitoring system which includes one or more cameras to determine a three dimensional (3D) model of a person; a means to detect a dangerous condition based on the 3D model; and a means to generate a warning when the dangerous condition is detected.

This invention may not always be accurate in identifying a fall event due to the infinite number of combinations of body and limb movements that a person can go through during a fall. This could result in numerous false alarms that result in unnecessary responses by paramedics or friends. These false alarms could result in desensitization of responders and their failure to urgently respond to a real fall. Similarly, false alarms could create the sense that the system is nuisance to the point that he user simply turns it off and loses detection of a real fall event. This system must have cameras in all rooms and yard to provide ubiquitous monitoring of a persons' environment. Additionally, furniture locations could result in several cameras being needed to ensure that there are no blind areas to the monitoring system. This could become unaffordable for people in need of fall monitoring. Suspicion of the where imagery could be seen could result in some modest people declining its use in the bedroom, dressing areas, or the bathroom which would decrease its practical utility.

U.S. Pat. No. 8,032,833 describes a method and system for providing a user interface for a user to discover and control devices that are currently connected to a network, such that at least one of the devices performs steps by: (a) obtaining information from one or more of the devices currently connected to the network, the information including device information; and (b) generating a user interface description based, at least, on the obtained information, the user interface description including a reference associated with the device information of each of the devices currently connected to the network, such that the reference includes at least one link to information contained in the devices currently connected to the network. As such, a user interface can be displayed using the references in the user interface description, for controlling the devices currently connected to the network.

This invention does not support meta data that fully specifies the device capability to the level required to adequately understand and use the data.

### Data and Information Processing:

U.S. Pat. No. 8,038,614 discloses a method for establishing baseline data for normalization in a physiological monitoring system which captures images of an individual in order to monitor the well being of the individual, including establishing the identity of an individual; assembling a set of personal data associated with the individual, including semantic data; using the semantic data to identify at least a portion of the wellness parameters to be monitored; acquiring images of the individual during separate capture events to establish baseline image-based wellness parameter data and baseline capture condition data for the individual and; using the baseline wellness parameter data and baseline capture condition data to monitor the physiological condition of the individual.

U.S. Pat. No. 8,038,615 discloses a method for inferring the physiological condition an individual using a physiological monitoring system including providing a set of personal data regarding the individual, the personal data including wellness parameter data or semantic data or both, wherein the wellness parameters correspond to known physiological attributes for the individual; acquiring physiological data of the individual and associated capture condition data, during multiple capture events; calculating wellness parameter data for the individual, using the physiological data and the capture condition data; analyzing the wellness parameter data to identify changes in the wellness parameter data; and inferring the physiological condition of the individual from the changes in the wellness parameter data in view of the semantic data.

The two inventions above, U.S. Pat. Nos. 8,038,614 and 8,038,615, are limited to health monitoring as enabled by capturing and assessing physiological information about a person from devices. It does not address monitoring for emergent, time critical events nor the response to such events. The system only addresses problems that can be inferred from physiological data, not mental issues such as depression. Additionally, the system does not describe the actual software architecture running the inference engine. For each application running on the inference engine and for each piece of hardware that is part of the invention, software must be written to make the data from the hardware devices compatible with the specific algorithms. Similarly, the software algorithms running in the inference engine must all have software written tailoring them to be able to use data from each data source. As sources change, the algorithms may potentially require new tailoring software.

Assessment of the Current State of the Art in the Fields of this Invention:

There are currently numerous environment and physiological monitoring devices and numerous inventions that create systems of monitors for physiological and environmental data. Some are focused on hospital settings, some on clinic or kiosk settings, and some on home settings. Many include connection to remote monitoring facilities and use video teleconferencing as part of the communications medium between a user and either the remote monitoring facility or a professional medical provider.

None provide the user with ubiquitous monitoring of a person and the environment for emergent as well as potentially emergent conditions with corroboration of detections and robust switching to back-up capabilities in the event of faults or connectivity loss. None provide for video connectivity to family, friends, and non-professional caregivers for the purpose of maintain good mental health.

All art relies on software for data processing and assessment. All neglect to address improvements on the state of the art of software architecting and creation of schemas. The result is that better monitoring technologies are finding their way to the market place but technologies to integrate new hardware and software devices into systems, and assess data has stagnated. As a result, software writers must still write software that is potentially unique or proprietary for each system arrangement because of the lack of standards in both hardware and software.

### SUMMARY OF THE INVENTION

This invention is a process and system to support safe, assisted, independent living. The invention also supports the use of hardware and data processing to create information in the home and workplace that generally increase quality of life or increase productivity of the user, increase utility and productivity of devices in the user's home environment, or increase quality of the environment. The invention's process is to create ubiquitous monitoring of the invention user's activities, physiology, and environment (the facility such as the home or work place and devices, utilities, and appliance); move information from monitoring and sensing devices to computers and analyze it; act on the information in a prioritized manner to address emergent events and potentially undesirable conditions; and then facilitate living and improve quality of life. The ‘matter’ of the invention includes sensors, devices, appliances, computers, data storage, and remote monitoring and ancillary locations.

The invention uses a system of integrated, networked hardware and software. The software modules run on computers that interface with monitoring and control hardware devices in homes, work locations, remote monitoring centers, and at ancillary locations. The invention includes the interfaces with users and parties at ancillary locations. The invention includes the software architecture and schema called Adaptive Scalable Plug&play Infrastructure for Responsive Engineering (ASPIRE). ASPIRE includes the following critical elements used to embody this invention: the Common Data Dictionary, a standard for writing Machine-readable, Self-describing Data Sheet (MSDS) schemas, structured query, and libraries of Application Programming Interfaces (API). These elements are defined in the definitions section. The structured query and API implementation methods are proprietary information owned by PnP Innovations of Albuquerque, N. Mex.

The invention software is an architecture of ‘middle ware’ applications that support the movement of data messages between hardware and software devices in the network. The invention middle ware applications enable hardware and software applications, when written to conform to the inventions' MSDS schema, they immediately identify themselves to all elements on the network, send information to users of data that they produce, and receive the optimum data available to meet their needs.

The invention takes a unified approach to describing data and satisfying the described interfaces, and ensures that data exchanged between modules in the system remains interpretable irrespective of the developer providing it or over the progression of time. The invention describes all data interfaces and data points in the system in “atomic” terms. For example, to capture blood pressure information and analyze it, the invention defines a “kind” of data item called “BloodPressureSystolic” that universally represents a well-known biometric measure. Formal definitions further augment the interpretation of this data by using modifying metadata such as “Measured” or “UpperLimit”. The shared ontology or dictionary of common descriptive data terms (Common Data Dictionary) including these types of definitions must be used to develop the descriptions of devices. By referencing the dictionary when developing software modules or hardware devices for the system, interoperability is guaranteed.

This invention characterizes software and hardware as being self-describing to a network, that is, the software or hardware element announces to the system what it is, what services or support it needs, and what services or support it can provide the hardware and software elements already in the system. This invention describes the usage of the ‘self-describing’ information, a data schema that must be followed for the information to be interpreted by a compliant system. The invention provides examples of the use of this data schema, but does not explicitly define the schema or the contents of the Common Data Dictionary. Both are necessary to enable the invention and must consequently be publicly distributed or users must create their own.

This invention describes specific software elements that perform the necessary functions of managing the information coming from the software and hardware elements and acting as a traffic director to control where information is processed and what information is passed between hardware and software elements. This capability enables the system to act as a networked system of systems, that is, processing to perform any function can be done on any computer in the system that is on the network. The processor has the ability to receive information from and send information to anywhere in the system, on the network. The invention further describes the software elements that monitor the system hardware and software elements, and cause appropriate changes when elements of the system are intentionally or unintentionally changed.

In summary, of the above capabilities and attributes of this invention, the result is the ability to create systems from standard compliant hardware and software that can be put together and function immediately, without the need for specific drivers, adapters, or component level configuration. Capabilities to support people in their homes can be created to operate from computers in the home, in dedicated support centers, or from ancillary locations. Because the system has awareness of its configuration and status, it will automatically adapt to the modification of its configuration when the addition or removal of system support elements while maintaining its ability to support people in their homes. This increases the safety of people using the system.

### Improvements to Existing Art

This invention is an improvement upon the existing state of the art in the following ways:

As a single system, the invention provides ubiquitous monitoring of people in their homes to include all rooms, yards, garages, workshops, and outbuildings as desired by the user; to sense emergent conditions such as falls, fires, smoke, carbon monoxide build-up; monitoring of physiological data; and monitoring of activities. The invention creates responses at the lowest level to minimize the response time and cost of the service. The invention provides corroboration capabilities to maximize probability of detection of emergent events while minimizing false alarms (which increase costs and risk non-acceptance of the services by users). The invention provides increased assurance of service by continuous self-monitoring and fault detection, and automatic switching to back up monitoring centers and data bases in the event of unplanned loss of communications between users and monitoring centers. The invention uses video conferencing to increase the effectiveness of medical treatment of people potentially needing help. The invention goes further to use video conferencing to maintain good mental health by connecting people to family, friends and non-medical caregivers.

The use of ASPIRE's CDD, software schema, and API libraries enables the invention to be tailorable to every user's specific needs, i.e., specific devices and software capabilities can be rapidly composed to meet the specific needs and desires of the invention user which provides the greatest benefit for the least cost. The nature of ASPIRE is to create a data rich environment which facilitates fault tolerance, corroboration of information, and the ability to create capabilities more focused on a user's specific needs or desires. Additionally, the use of ASPIRE enables rapid integration of hardware and software components so that users can quickly benefit from technology developments.

Ubiquitous monitoring and emergent event response: Many embodiments of current art sense only at specific locations in the home such as in a special chair where physiological data is captured or in front of cameras that image to enable assessment of physical and mental health. This invention uses relay's in each room to collect data from physiological and environmental sensors, and pass them to the user's main processor. Additionally, the fall detection and help button device are worn continuously in all rooms of a user's home or work area. When an emergent event is detected by the processor, the voice queries from the main processor, “Do you need help,” and follow-up queries from the CSC are broadcast through Room Boxes in all rooms.

Response at the lowest level in the system: Many embodiments of current art collect data and send it to remote locations for assessment and action. This invention is able to perform triage, i.e., users can install software applications on the user's main processor (in the home) and have interaction occur between the processor and the user without involving the Central Support Center (CSC). Additionally, personnel at the CSC orchestrate support at the lowest level of professional or non-professional medical and health caregiver. This is critical to making the invention affordable to the broad majority of potential users.

Event corroboration: Many embodiments of current art do not address verification of events or conditions. This invention will take inputs from various passive sensors to corroborate an event or condition. For emergent events, it will use its speech generation capability to query user's as to their need for help. This is followed up by operator calls from the CSC. This feature of the invention is enabled by the Rich Query and the Data Centric Streams Layer features of ASPIRE (discussed later). In the case of falls, after the fall detection bracelet detects a fall, it monitors for subsequent motion indicative of normal walking. Lack of motion is further corroborated by data from motion sensors in the Room Boxes. The significance of this invention's corroboration capabilities is that events and conditions can be more accurately identified. This increases the value of the invention to the user because emergent events or significant conditions are more reliability detected and false or ‘nuisance’ alarms are avoided. False alarms are especially significant as they desensitize the user and possibly personnel at monitoring centers to potentially dangerous events and conditions.

Self-monitoring, fault detection, and automatic switching to back-up capabilities: Many embodiments of current art do not address self monitoring or the use of back up capabilities. This invention uses ASPIRE to enable monitoring of all components and nodes in the networks created to support users. The CSC is made aware when a device fails in a user's home. The Rich Query and the Data Centric Streams Layer features of ASPIRE enable the ability of applications to automatically find and use other sources of data if they are available on the network. Additionally, the CSC is able to remotely repair software on the user's computers. Similarly, when the user's main processor detects lost contact with its CSC, it will automatically search for and connect with alternate CSC to continue that element of support. CSC's also automatically find back up data storage locations when contact is lost with their primary data storage devices.

Use of video conferencing: Embodiments of current art employ video teleconferencing across a range of uses but most typically do not leverage this technology to widen the medical, emotional, and mental health support base by enabling users to have video conferencing capability with semi-professionals and non-professionals such as non-skilled caregivers, family, and friends. This invention uses video teleconferencing in both structured and un-structured manners. In addition to connecting users to CSC personnel who can either handle the situation or transfer a call to a medical professional, it also enables users to ‘dial direct’ to family and friends from their own computers. This gives them a sense of greater control of their lives as well as helping keep the system affordable.

The invention's use of ASPIRE is what brings the greatest improvement to the field of plug and play software and to the field of systems that facilitate independent living and systems that perform health and environment monitoring and intervention.

Embodiments of current art require custom software to connect new hardware or software devices to a system. The use of ASPIRE in this invention minimizes or eliminates this activity.

The software that automatically finds sources of data needed by other hardware or software applications is the ‘Rich Query Engine.’ The software that finds the best data to use by applications is called ‘Data-Centric Streams Class.’ The ‘Rich Query Engine’ and ‘Data-Centric Streams Class’ software together create the ‘play’ attribute of this invention. In this invention, ‘play’ means that a hardware or software component is able to immediately function when added to the system or network. That is, there is no need to create custom software that adapts the new hardware or software component to the existing system. This is the innovation and improvement of software in this invention.

There are numerous results and benefits of compliance with the standards and use of software modules in the framework described above. One revolutionary benefit is that software modules can immediately be adapted from one system or network to another that is running hardware made by completely different vendors and different software modules. A second benefit is that the framework applies well to systems ranging from simple to complex. A third benefit is that software modules will remain compatible with changes of other modules in the system because the software interfaces are at a fundamental level of data definition.

The Rich Query feature of this invention is the key to being able to integrate new hardware into the network or install new software applications onto a processor, and have these devices immediately performing with no need for specially tailored interfaces. The Rich Query engine automatically performs the searches for sources of data when applications send out needs for data. The Rich Query engine automatically updates the sources so as to keep track of new sources of data or when sources of data are lost.

The use of data centric streams has two significant advantages in manage multiple sources of similar data. First, reliability of the system is increased because in systems where there are multiple ways to get the same data, the software makes one source as primary and automatically defaults to a second source when the first becomes unavailable. Second, the use of data centric streams precludes the requirement to write custom software to increase reliability of all software functions.

The Data-Centric Streams Class software feature works with the Rich Query engine but takes the next step of selecting the best source of data to be used by an application and giving it to the application. Working together, the Rich Query engine and the Data-Centric Streams Class work to increase the reliability of the system created by a user of the invention. Should a source of data become un-available, the system will automatically find an alternate source of data and continue functioning. This continuity of operations is critical is for the safety of a person using the invention to provide oversight of a person in his home.

### BRIEF DESCRIPTION OF THE DRAWINGS

FIG. 1 is the block diagram depicting the main components of the invention.

FIG. 2 is the block diagram depicting the main components of the user environment.

FIG. 3a is the block diagram depicting the home user interface screen display showing video conferencing call options

FIG. 3b is the block diagram depicting the home user interface screen display showing an example of a calendar and medications reminder

FIG. 4 is the block diagram of the main components of the Room Box

FIG. 5 is the block diagram of the fall detection sensor and ‘help’ button device

FIG. 6 is the block diagram of the main components of the combined support center

FIG. 7 is the block diagram of the main components in ancillary locations.

FIG. 8a depicts the functional software blocks that constitute the software invention with an arrow depicting the passing of subnet domain registration data to the local IP Stack register.

FIG. 8b depicts the flow of IP domain data to the processor manager from the local IP Stack.

FIG. 9a depicts the sending of interrogation messages from the Network Manager to devices on its immediately visible sub-network.

FIG. 9b depicts the Network Manger sending sub-net routing tables and address assignments to nodes.

FIG. 10a depicts the Data Directory sending the local Processor Manager a message that describes the type of component (Data Directory) and its address.

FIG. 10b depicts a Processor Manager broadcasting a core component registration message to its sub-network nodes.

FIG. 11a depicts a component sending a ‘Hello’ message to all Data Directories to assert its presence on the network and identification.

FIG. 11b depicts a Data Directory acknowledging a ‘Hello’ message from a node or component.

FIG. 12a depicts the Data Directory requesting a data description from a re-registered or newly registered component.

FIG. 12b depicts a hardware device on a sub-network sending its component data description to a data directory.

FIG. 13a depicts an application requesting of and receiving a list of data directory components from the processor manager.

FIG. 13b depicts an application sending a ‘Hello’ message to a data Directory with unique identifiers for the component and data descriptions.

FIG. 14a depicts the Data Directory acknowledging a ‘Hello’ message from an application.

FIG. 14b depicts the Data Directory sending a ‘Request Data Description’ message to an application.

FIG. 15 depicts an application or hardware device sending its data description to the Data Directory which stores it in its Data Storage.

FIG. 16a depicts an application or hardware device sending a ‘Structured Query Request’ to the Structured Query Engine.

FIG. 16b depicts the Structured Query Engine looking into its associated data base for matches between data and data requested by an application or hardware device.

FIG. 17 depicts the Structured Query Engine sending matches of data requested by an application or hardware device.

FIG. 18a depicts an application or hardware component sending a data subscription request to another application or hardware component.

FIG. 18b depicts a data provider sending data messages in accordance with subscription request.

FIG. 19 depicts a subscriber sender a data provider a data message lease renewal or modification.

FIG. 20a depicts a consumer application sending a Data-Centric Streams Class the management policy for multiple data query matches.

FIG. 20b depicts the Data Centric Streams Class passing a structured data query to the Data Director's structured query engine.

FIG. 21a depicts the Data-Centric Streams Class issuing data subscriptions to data providers.

FIG. 21b depicts the Data Centric Streams Class receiving data from providers and only sending results to the consumer in its specified format.

FIG. 22 depicts the block diagram of the invention's processor software architecture

FIG. 23 depicts the block diagram of the Room Box software architecture

FIG. 24 depicts the block diagram of the fall detection device and help button software architecture

FIG. 25a is the block diagram of the Nominal Combined Support Center, PBX, and User's Main Home Processor Operations

FIG. 25b is the block diagram of the Combined Support Center, PBX, and Main Home Processor Operations during Internet Connectivity Interruptions

FIG. 26 depicts the integration architecture of a non-ASPIRE compliant software application

### DETAILED DESCRIPTION OF THE INVENTION

### Definitions:

All figures in this disclosure depict the invention in best mode contemplated by the inventor at the time.

Application Programming Interface (API): the format for describing data from a software application or hardware that makes it is useable by the invention. It is used by software application writers or hardware inventors.

Applique Sensor Interface Module (ARM): The hardware or software device that converts the transport layer around data as it moves between systems working on different transport standards. An ASIM bridges a device that exports or consumes data written to a set of transport specifications to a network operating on a different data transport standard.

ASPIRE: The name of the invention software architecture and schema for writing architecture-compatible Machine-readable, Self-describing Data Sheets (MSDS). ASPIRE is the acronym standing for Adaptive, Scalable, Plug and play Infrastructure for Responsive Engineering.

Atomic data: This is the most primitive type or smallest piece of data that is supported by the processor itself.

Common Data Dictionary (CDD): The CDD is a reference standard used by writers of software for this invention. It is a comprehensive listing of the names of data and their associated definitions. Its purpose is to prevent confusion or redundancy. It allows a software writer to be able to write an application that provides data to another set of applications, written by a different software writer, that is able to use the ASPIRE architecture to find and use the data automatically, with no coordination between either software writer. An example is temperature. The CDD would define ‘temp’ as the expression of temperature. It would be the only recognized term whose definition is temperature as opposed to other notations such as ‘t,“T,” Temp,’ etc. When software writers are writing applications that need information, or data, from the system, they use the definition from the CDD to describe what they need. Similarly, when software writers write applications that create information or data, they describe that data by the definition in the CDD. For this invention, the users will create their own CDD that contains terms to describe data needed by the software applications of the system. As the system evolves and grows with additional software and hardware added, the CDD will have to be expanded to incorporate dress new terms.

Core Components: The minimum three essential software devices and components that enable ASPIRE to allow self registration of hardware and software components, and enable automatic functioning of these components. The Core Components consist of the Network Manager, the Processor Manager, and the Data Directory which includes its database, the Rich Query Engine, and the Data-Centric Streams Layer.

Data-Centric Streams Layer: This is reusable software that resides as a library on processors which allows a application to make a data query and it manages multiple providers of that data so as to provide the best data for the application.

Data Dictionary: this is the term for the software data base that acts as a repository of information for devices conforming to the invention's plug and play protocols.

Device: This term describes a hardware article or a software module that performs a function at some level of the system.

Machine-readable, Self-describing Data Sheet (MSDS): data associated with specific hardware or software modules that contain the following information about the hardware or software module: name of the device, function, power needed, information needed from other devices or software modules, format of information of data coming from the device, and physical specification of data from the device.

Message Delivery Service: This is a message delivery service, such as an internet protocol (IP) stack or message queue, implemented as a software function located at a processing node that acts as a mail sorter or switch board operator and moves data between applications, and between applications and data storage locations.

Metadata: Metadata is generally described as data about data. In this patent, the term metadata means the technical description of data and is usually describing data at its most primitive level. For this invention metadata is almost always referring to physical quantities. For example, information about the heat measured by a device is described in degrees, power of an electrical current is described in volts, and units of length are described in metric units such as centimeters or kilometers. Besides quantities, metadata can also describe other attributes about the data that can facilitate a better understand the utility of the data by software applications. Examples of the other attributes could be the accuracy of the data; the rate of change of data, i.e., degrees per second; or the rate that data is produced or captured, updated in a data base, or the rate that it is delivered.

Network: The means by which hardware and software elements of the invention's architecture exchange data. Components on the network (devices or applications) are reachable by any other component on the network. Networks created in the context of this invention may be hierarchal in the way that hardware and software are organized, i.e., there may be logically-organized levels or sub-domains of functionality that move data upwards and downwards.

Network Manager: For this invention, a Network Manager is a software component, located with a processing node, which understands how to move messages between applications on the processor and destinations residing on the network to which it is attached. In lay terms, this registration is like a rural post office telling a state's main post office exactly what zip codes are served by the rural post office. Just as the main post office knows which rural post office to send mail based on specific zip codes, Network Managers installed on a processor allow that processor's central routing authority (typically an IP stack, but not constrained to be so) to know which subnet should receive an outgoing message based upon its destination address.

Processor manager: This is an application that is common to all processors in the invention's wide network. A processor manager maintains the knowledge of sub-network domains and, as we will see in later paragraphs a list of “core component” addresses.

Rich Query: This is the name of a software utility class inherent in the invention that provides the following capabilities. First, software applications may express their data needs in succinct, unambiguous, and highly descriptive language conforming to a Common Data Dictionary. The request is sent to a Rich Query Engine which scans a database containing the collective descriptions of all components that have joined the system. Secondly, the software application can express characteristics of the needed data with richly described attributes to which the data should conform (e.g. ‘deliveryRate’ must be at least once per second, accuracy must be within 0.5 deg Fahrenheit). Thirdly, the application can specify that its metadata desires be exact or conditional (such that any match greater than the value passed is acceptable, for example). Fourth, the application requests the return of a list of metadata values that can be used to better understand and prioritize the matches that are returned. This metadata may be used by the application to (as an example) decide which message source to favor. If the preferred message source becomes unavailable for any reason, the application can elect to immediately switch over to a backup.

Rich Query Engine: This is the software utility that interfaces with a Data Directory's database to perform searches for suitable sources of data based upon expressions delivered by applications using the Rich Query Software Class.

Rich Query Specification: A message containing a description of data desired by an application to fulfill a monitoring or processing function. A Rich Query Specification consists of an atomic kind, a standard interface name, and an optional set of descriptive metadata to further augment the desired characteristics. The format of the specification is governed by the Common Data Dictionary.

Router Table: This is the name for a data table that describes the associations between the ports of each router and other routers or nodes directly visible to them on the network. A complete collection of Router Tables delivered to any node on the network allows that node to compute the route to any destination. Router Tables may be updated by a Network Manager component if links in the network are added or removed or if individual nodes come and go.

How to Build this Invention

Below is the description of how processors and specific hardware devices work in a networked fashion to provide monitoring and control of home and work places. Following this section is a detailed description on how to create the software to work in an integrated fashion to support functioning of hardware devices, gathering and processing of data, and supporting human interfaces at various places in the network.

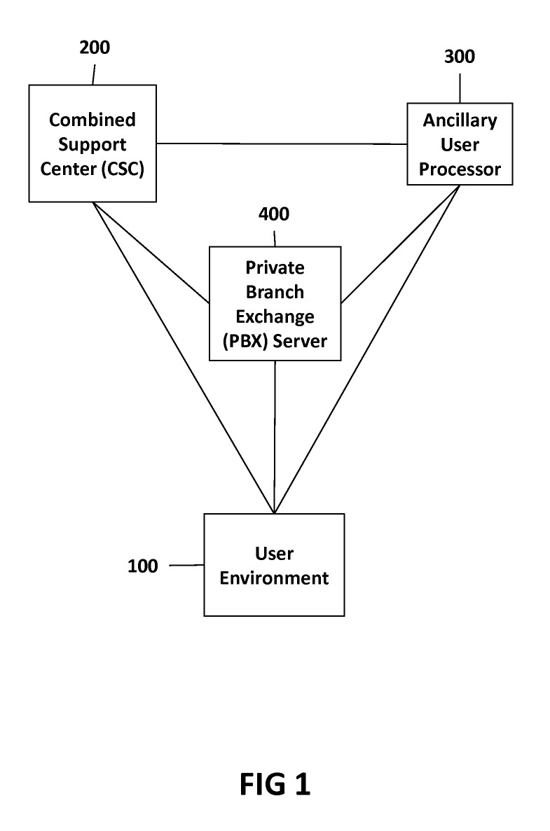

FIG. 1 is a block diagram representing the best mode and highest level view of major components of the invention in one of many logical embodiments. These consist of the User's Environment 100 (which can be home work); or a special facility, or freestanding hardware and software of this invention that takes advantage of the inventions capabilities; the Central Support Center (CSC) 200; the Ancillary User 300; and the Private Branch Exchange (PBX) server 400 which brokers video teleconferencing connections between the major components of the invention.

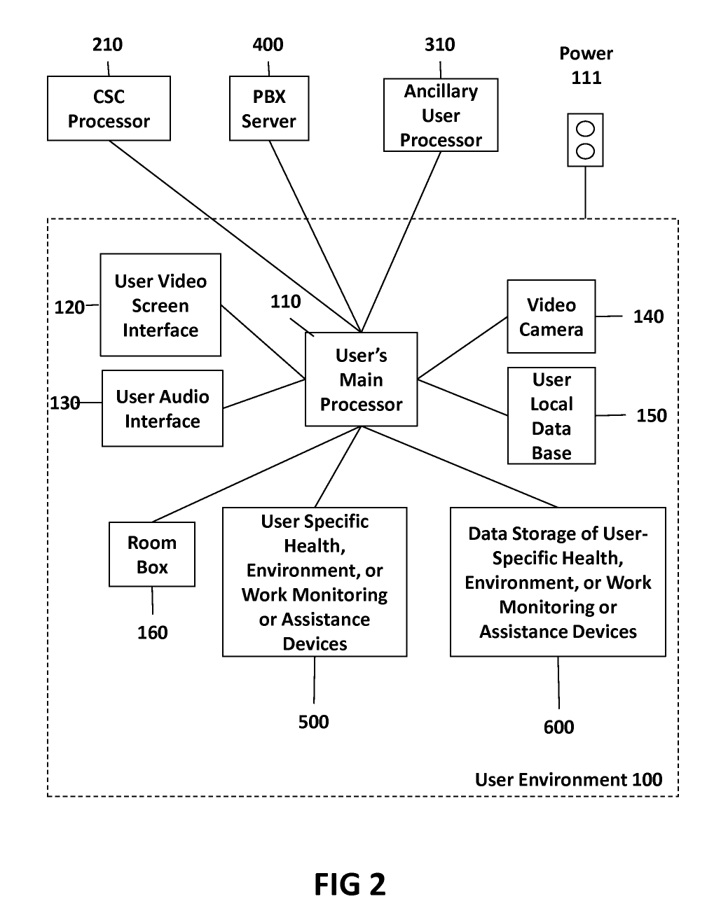

FIG. 2 depicts what is connected to the User's Main Processor 110 which is located in a user's home or work environment, or special facility mentioned above. The User's Main Processor 110 interfaces with ten sets of devices: the User Video Screen Interface 120; the User Audio Interface 130; the Video Camera 140; the User Local Data Base 150; the Room Boxes 160; the external CSC Processor 210; Ancillary User's Processor 310; the PBX 400; User Specific Health, Environment, Work Monitoring or Assistance Devices 500; and Data Storage of User-Specific Health, Environment, Work, or Assistance Devices 600. Power 111 is through a uninterrupted power system to temporarily support commercial power outage situations.

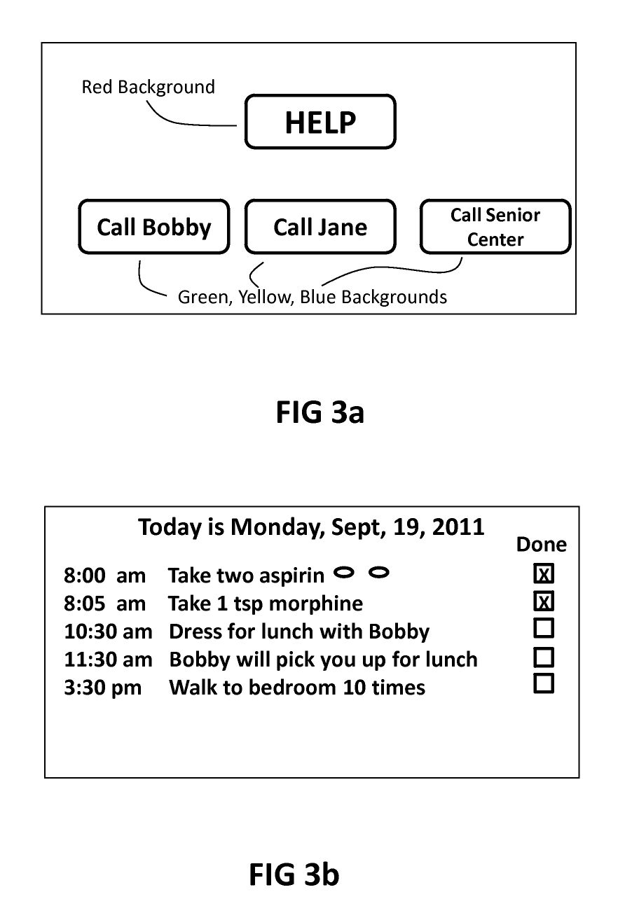

Information from the User's Main Processor 110 is displayed on the User Visual Interface 120 such as a computer monitor or television. The computer monitors and television screens can be touch activated, or interfaced with computer mouse for keyboard. FIG. 3a depicts a typical screen that the user would use see when the user would want to make a call. Large, virtual buttons are labeled to make it easy for the user to choose who he or she would like to call, and colors are tailorable to the user's needs. This screen would be displayed as the screen saver or when the user says ‘help,’ or ‘call Bobby,’ or one of the names on the display. An alternative screen saver display is a calendar or reminder checklist that is shown in FIG. 3b. This calendar and reminder list would be created by a person who had specific permissions to make entries. Times for events would be displayed and a check-off box would be used to assist the user to know what events remained for the day. For certain events or activities, images will be displayed to help the user understand exactly what needs to be done. The figure shows an image of pills but this could be photographs and could be used to help the user remember what items of clothes to wear or what items of food to prepare and consume.

The User's Main Processor 110 also interfaces with a speaker and microphone arrangement, the User Audio Interface 130, to support video teleconferencing; and to support transformation of text information, questions and commands generated by the User's Main Processor 110 to audible information; and to support the transformation of the user's speech into text onto the User's Main Processor 110 or into answers to questions and commands using speech recognition software recognizable by the user's processor.

The User's Main Processor 110 also interfaces with a Video Camera 140 to capture imagery of the user and his environment. The video camera can be built into the same unit as the User's Main Processor 110 or it can be stand alone using a wireless or wired connection to the User's Main Processor 110.

Additionally, the User's Main Processor 110 interfaces with the User's Local Data Base 150. This data base contains information that is used by software running on processors on the network to support the home and work environment monitoring, assistance to and interaction with the user as well as external locations and people.

Furthermore, the User's Main Processor 110 interfaces with Room Boxes 160 that are distributed throughout the home or workplace. The Room Boxes 160 use a wireless radio or wired connection to transfer data between the User's Main Processor 110 and the Room Boxes 160. The number of Room Boxes 160 in a home or workplace is based on the number of rooms to be monitored. Additionally, Room Boxes 160 can be used to relay information from other Room Boxes 160 to the user's processor in the case of large homes and workplaces, or environments.

The User's Main Processor 110 is connected to devices that capture in real time data from User Specific, Health, and Environment or Work Monitoring or Assistance Devices 500. The User's Main Processor 110 also moves information to or retrieves data from external Data Storage of User Specific Health, Environment, or Work Monitoring or Assistance Devices 600. The software mechanisms for storing or retrieving this data are described later in this section.

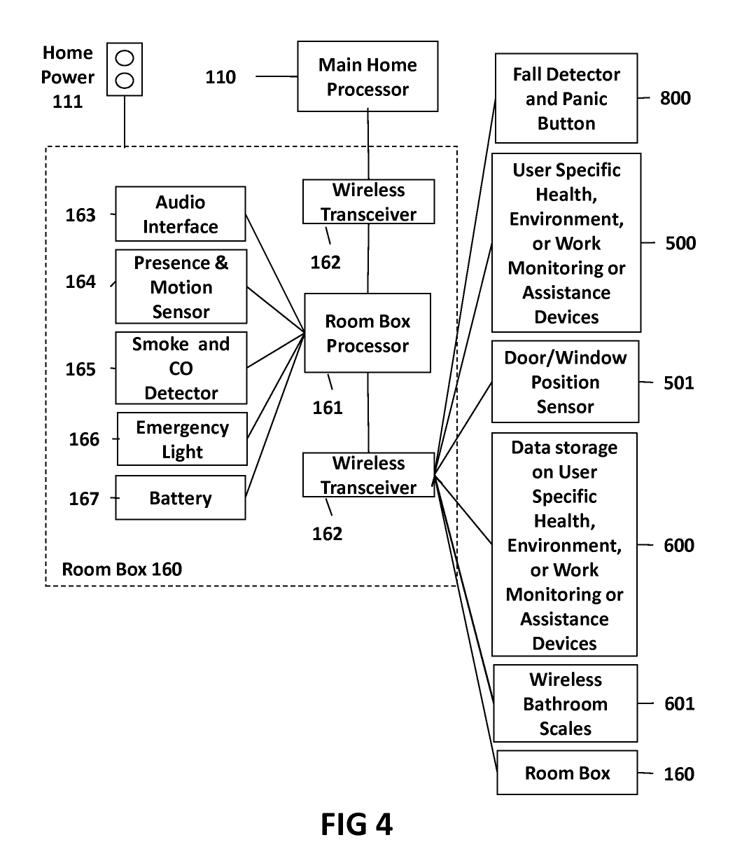

The Room Box 160 as depicted in FIG. 4 hosts a Room Box Processor 161 capable of running software to support user needs. The Room Box 160 may host a network manager, data director, and processor manager if these are needed to support the managing of the network set up to meet user needs.

The Room Box Processor 161 is connected to an Audio Interface 163 that is able to relay speech and sounds between the User's Main Processor 110 and the user through built-in speakers and microphone. The interface also has a built-in microphone that can receive speech from the user and environmental noises. The user's main processor has the capability to act as a relay between a Room Box 160 and the CSC 200 to support conversation between a user needing help and the operator on duty at the CSC 200.

The Room Box 160 has a human Presence and Motion Sensor 164 that is able to detect motion that can be used to corroborate fall indications from the fall detection device. The motion sensor can also used to provide knowledge of user movements (both temporal and locations) to the User's Main Processor 110 for storage as data or relay to the CSC 200.

The Room Box 160 also has a Smoke and Carbon Monoxide (CO) detector (in environments conducive to CO detection) 165 that sense smoke and CO, and transmit this information to the user's main process for notification to the user of an emergent situation, and the CSC 200.

The Room Box 160 has an Emergency Light 166 that is activated for a short period of time in the event of a signal from the Fall Detection Processor 810 indicating a fall, activation of the help button 860, or verbal request for help from the user.

The Room Box 160 is connected to home or work place Electrical Power 111. The Room Box 160 also has a Battery 167 to enable functioning for short periods of time during outages of commercial power.

In addition to wirelessly communicating with the User's Main Processor 110, the Room Box 160 also wirelessly or in a wired configuration connects to a range of devices in the room.

These include the Fall Detection and Help Button Device 800, Physiological, Health, Environmental, Work, and Assistance Monitoring Devices 500 that transmit data and information immediately upon capture, or store and transmit data, post activity, to Data Storage on User Specific Health, Environment, or Work Monitoring or Assistance Devices 600. An example of a device that immediately transmits data is a Door/Window Position Sensor 501 that transmits a change as soon as it is detected by the device. An example of a device that sends stored data is a Wireless Bathroom Scale 601 that takes three readings in a row, averages them, and then sends the average data to the User's Main Process 110 or for forwarding to a storage location.

Each Room Box 160 is able to wirelessly or by wired connection with other Room Boxes 160 within range pass information needed by elements of hardware or software on the network.

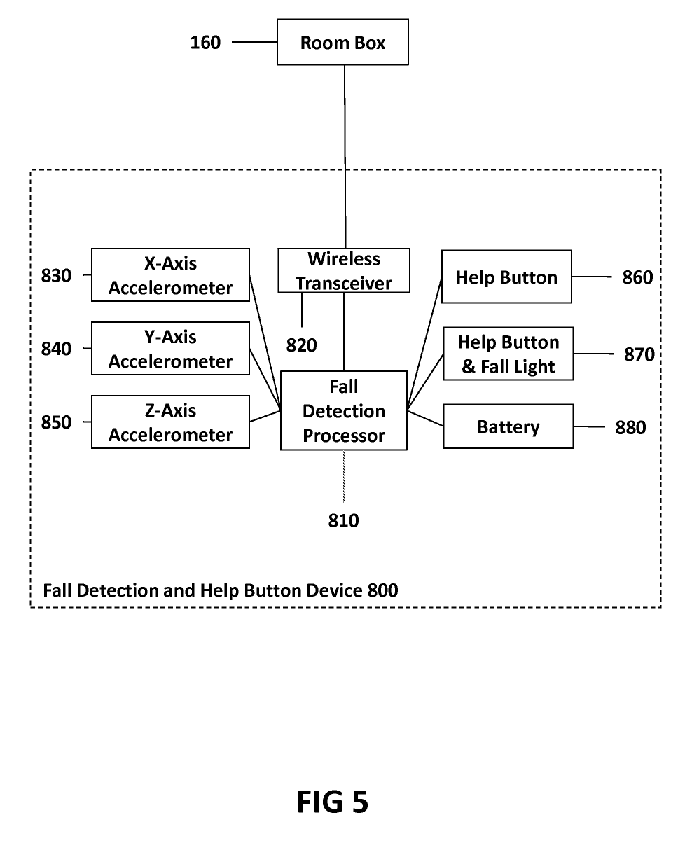

The Fall Detection and Help Button Device 800 are described in FIG. 5. The device is a circuit board on which are mounted a Fall Detection Processor 810 and three Accelerometers 830, 849, 850 that are measuring acceleration forces in the circuit board's X, Y, and Z axis. The accelerations and orientation of the accelerometers are used by software on the processor to detect indications of a fall. When a fall is detected, the processor sends a fall indication signal to the Room Box 160. This signal is forwarded to the User's Main Processor 110. A fall detection algorithms looks for subsequent motion from the fall detection devices accelerometers 830, 840, 850. It also queries the Room Box 160 for motion indicating walking, and verbally queries the user, “Do you want me to call for help?”

A ‘Help’ button 860 is mounted on the same circuit board as the above three Accelerometers 830, 840, 850. When the user pushes the button, the circuit board sends a signal to the Room Box 160 which relays the signal to the User's Main Processor 110.

When a fall is detected or when the help button is depressed, a small Help Button & Fall Light 870 in the Fall Detection and Help Button Device 800 illuminates to indicate detection of one of these signals and proper operation of the device.

The Fall Detection and Help Button Device 800 electronics are powered by a Battery 880.

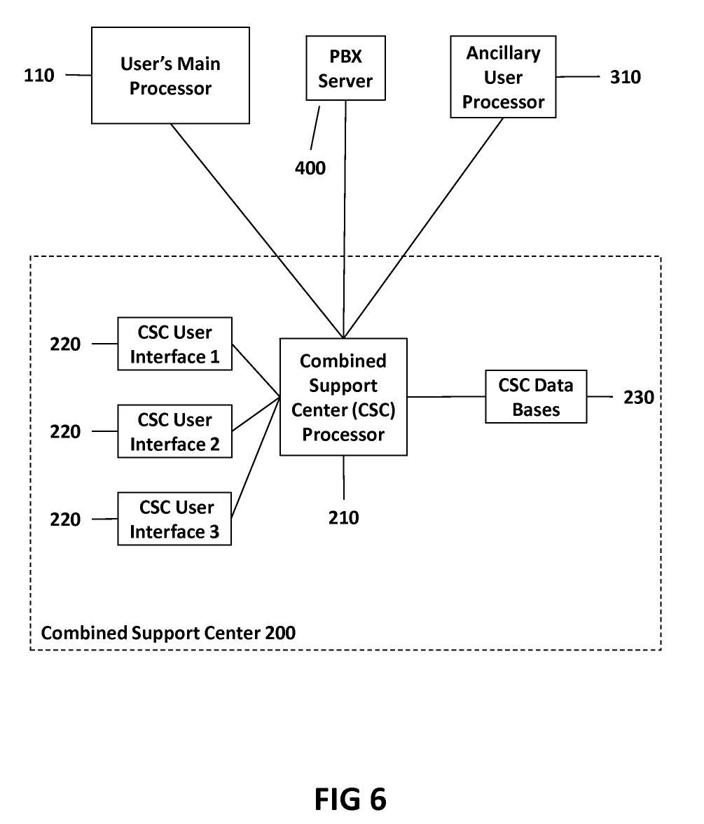

External to the user's environment is a Combined Support Center (CSC) 200 as depicted FIG. 6. The CSC Processor 210 is connected to the User's Main Processor 110, CSC User Interfaces 220, the PBX Server 400, and Ancillary User Processors 310, and CSC Data Bases 230 by internet to pass data and video. Telephone connection can be a back-up to internet. Video calls are brokered by the PBX Server 400.

The CSC is continuously staffed by human operators who are the first line of response to emergencies and urgent needs in the users' environment. The CSC Processor 210 interfaces with the operator by displaying information on User Interfaces 220. These monitors are used to monitor incoming calls and emergency signals; to display video conferencing call imagery; to enable call transfers; to access a listing of contacts and place video calls to them; to view specific client information that includes but is not limited to name, address, date of birth, sex, health conditions, medications schedule, diet needs, care plan, hospital discharge instructions, comments log (that captures interactions with the CSC operator, health providers, care givers, or diet/medication coaches), street map to home and layout of home, and displays specific user tailored instructions guiding the operator in their response to a call from the system user.

The user information is stored on CSC Data Bases 230 that are accessed by the CSC

Processor 210 to populate the operator screens.

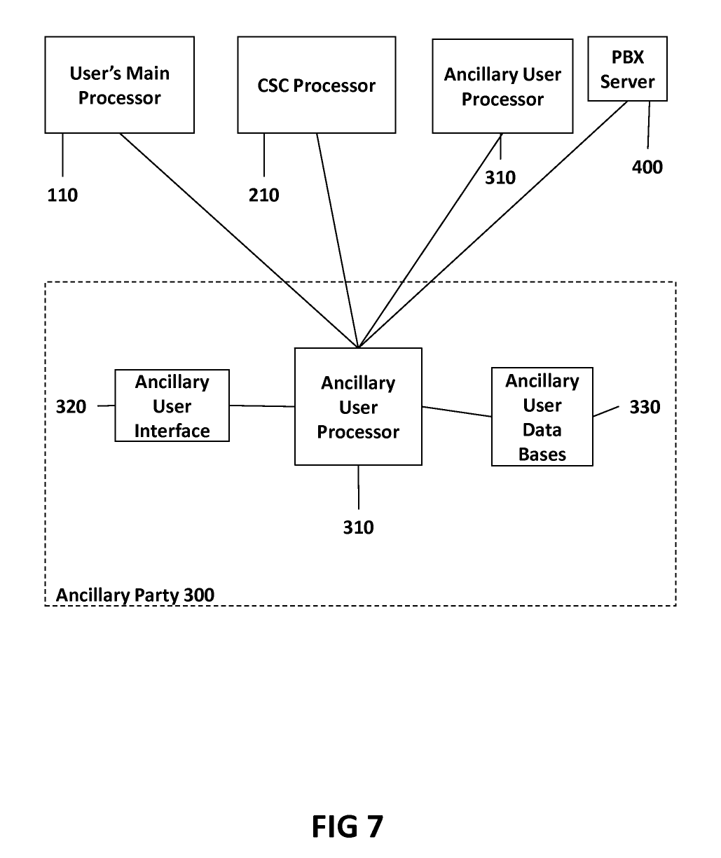

FIG. 7 depicts the components at ancillary locations. Ancillary parties are connected through their Ancillary User Processors 310 to the User's Main Processor 110, the CSC Processor 210 and other Ancillary Parties 300. The PBX Server 400 brokers video calls between Ancillary Parties 300 and other people on the network. Information from the Ancillary Party's Processor 310 is displayed on a computer monitor or television screen, the Ancillary User Interface 320. Data needed by the Ancillary Party 300 is maintained in the ancillary party's own, on-site Ancillary User Data Bases 330 or it may be located on User Local Data Bases 150 or the CSC Data Bases 230. Ancillary Parties 300 access data is enabled by the permissions granted them.

The software concept and architecture are described in the following paragraphs. All hardware associated with this invention is connected by software.

This section of how to build this invention switches to describing the software mechanisms and features that facilitate data interactions that comprise the plug and play feature set. These mechanisms and features enable the software elements to work in an integrated fashion to support functioning of hardware devices, gathering and processing of data, and supporting human interfaces at various places in the network.

The feature areas include the following: 1) Network discovery of devices, 2) Registration of devices, 3) Registration of applications, 4) Support of Rich Data Queries, 5) Data subscription management, and 6) Data-Centric-Stream management

The integrated functioning of these feature areas constitutes the plug and play capability that allows both hardware devices (such as blood pressure monitors, scales, etc.) and applications (such as trending and monitoring algorithms for client data) to join the system and interact without specific customization.

The invention leverages networking concepts to create a uniform addressing space for all components (whether they are pieces of hardware or software applications) so that they may freely message each other.

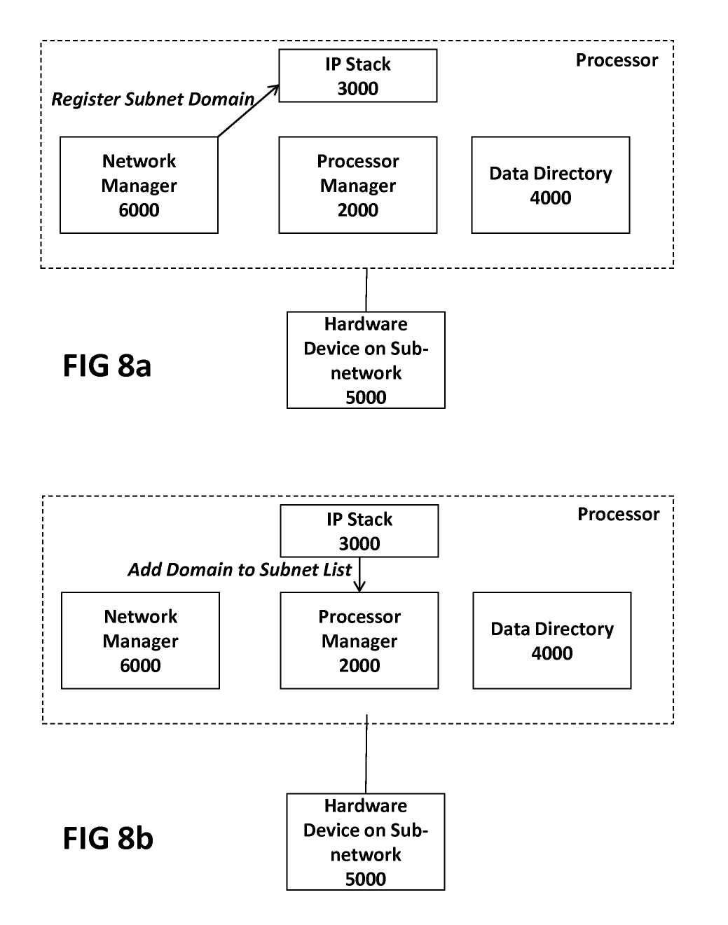

Network Discovery: The first feature area to describe is the discovery of devices. Devices may reside either on a local network as drivers that present an interface that complies with the message interactions described, or they may exist on a sub-network that is attached to a processing “node” such as a home computer, business workstation, laptop, or embedded microprocessor. FIG. 8a is the introduction to the functional blocks that constitute the software invention. This is a subset of the larger system and additional blocks will be introduced in later diagrams as needed.

In the case of any processing node used to support the invention there is a Network Manager 6000 supporting each sub-network that is attached to the Processor Manager 2000. There may be one or many sub-networks attached to the node to support different variations of devices (to include low-speed, simple devices that exchange very little data, and high-speed networks that support devices that communicate large volumes of data very rapidly). As each Network Manager 6000 is started as a process, it registers a sub-network domain with the local IP Stack 3000 on the processor. This allows the processor to understand how to route messages to devices that reside on the sub-network.

Once the Network Manager(s) 6000 have been started, the Processor Manager 2000 core module (as shown in FIG. 8b) detects the addition of a new IP domain and creates a new entry in a sub-network table. The Processor Manager 2000 is the common entity on all processors in the invention's wide network (the network reachable by any application or device installed in a home, business or care professional's support appliance) that maintains the knowledge of sub-network domains and, as we will see in later paragraphs, a list of “core component” addresses.

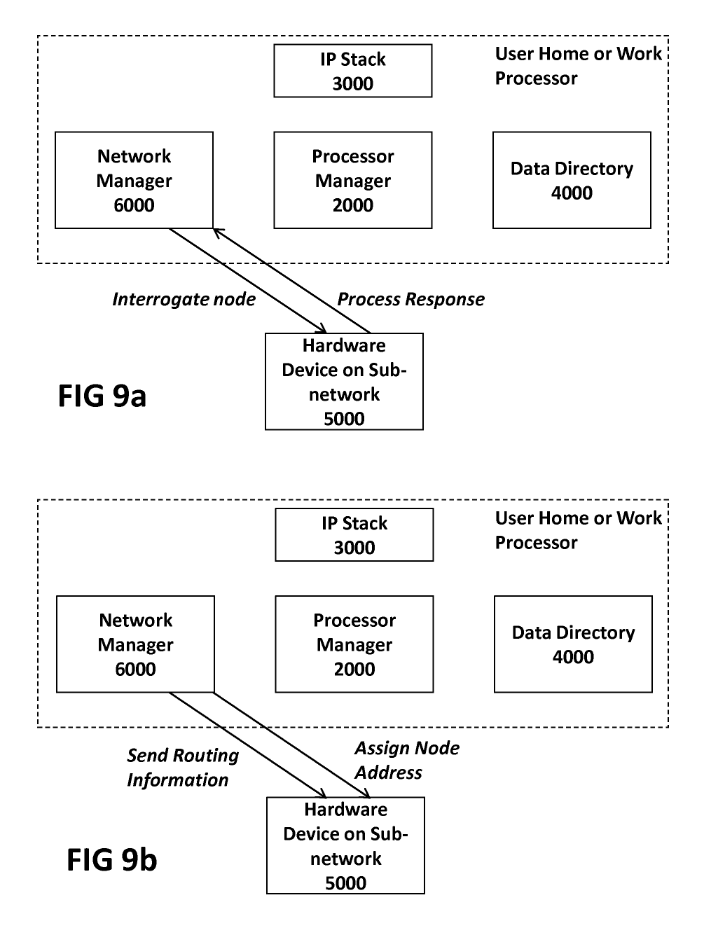

As soon as the Network Manager 6000 component is started, it will begin the process of detecting Hardware Devices on Sub-networks 5000 that are present on the sub-network. This is accomplished by sending an interrogation message that is understood by any device that it reaches (as shown in FIG. 9a). Interrogation is accomplished on a progressive basis, whereby the first devices reachable from the vantage point of the Network Manager 6000 are contacted first. Devices may be 1) routers, which branch to other routers or devices, or 2) devices, which are the terminating nodes of the network. In either case, the message returned (the interrogation response) is collected and used to determine the type (router or node). Continued interrogation is based upon the type encountered—a router indicates which of its physical ports are active and thus provide access to additional areas of the sub-network, while arrival at a node requires no further investigation.

When the Network Manager 6000 detects that all routers have been explored and all nodes have been reached, it utilizes either local assignment logic or unique identifier information provided by the routers or endpoints to build tables that describe the associations between each router and other routers (or nodes) that are directly visible to that router. These are referred to as router tables in following text.

Once all router tables have been constructed, the Network Manager 6000 distributes information to every endpoint reachable on the sub-network. The two pieces of information include 1) the router tables described in the previous section and 2) the assigned address of the node. This interaction is shown in FIG. 9b. Each of these items is delivered as a single message or message sequence using routing that may be unique to the sub-network that it services. Once these two pieces of configuration data have been delivered to all nodes, these nodes use the router tables to determine routing between any two nodes on the same sub-network by building a routing map from the router table information. This map is created from the perspective of each individual node.

As components are added to the network or removed from the network, Network Manager's 6000 detection mechanisms update router tables and redistribute them to all nodes. Depending on the sub-network, this is accomplished using heartbeat protocols or by firmware mechanisms built in to the routers themselves.

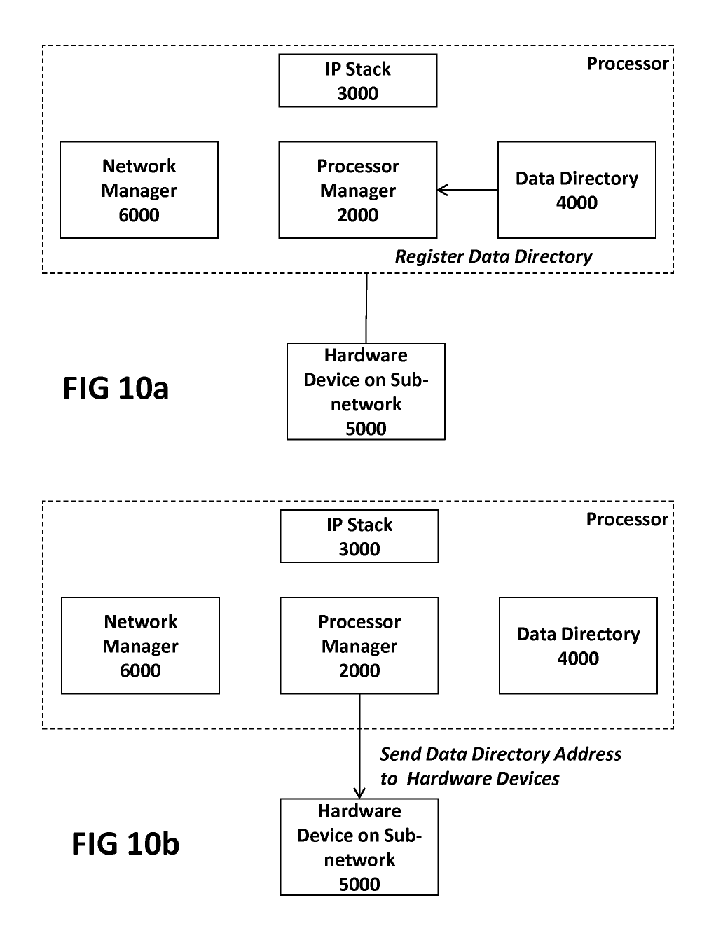

Component Registration: The second plug and play feature of the invention is the ability to register a component with a Data Directory 4000. The Data Directory 4000 acts as a repository (or database) of information for devices conforming to the invention's plug and play protocols. There can be one or many Data Directories 4000 in the system. When an instance of a Data Directory 4000 is created within the system it contacts the Processor Manager 2000 at a well-known address on the local processor (the processor on which both a Data Directory and Processor Manager are running), passing a message that describes the type of component (Data Directory) and its address (shown in FIG. 10a). Recall that the second function of the Processor Manager 2000 core component is to maintain a list of all core components.

When a Processor Manager 2000 receives a core component registration message, it propagates this message to all nodes on all sub-networks (as shown in FIG. 10b). This is accomplished by use of a broadcast address that is recognized by the Network Manager 6000. Receipt of the broadcast address by a Network Manager 6000 will result in that message being sent to all nodes reachable on the sub-network. Each node on the network, regardless of whether it is another processor or a hardware device, will have the equivalent of a Processor Manager 2000 installed to receive and process the core component message. This set of interactions insures that all nodes maintain a uniform view of the core components available within the system.

If core components are detected as unresponsive or are intentionally terminated, the Processor Manager 2000 on that processor removes the core component from the managed list and propagates the deletion to all other reachable nodes in the same manner

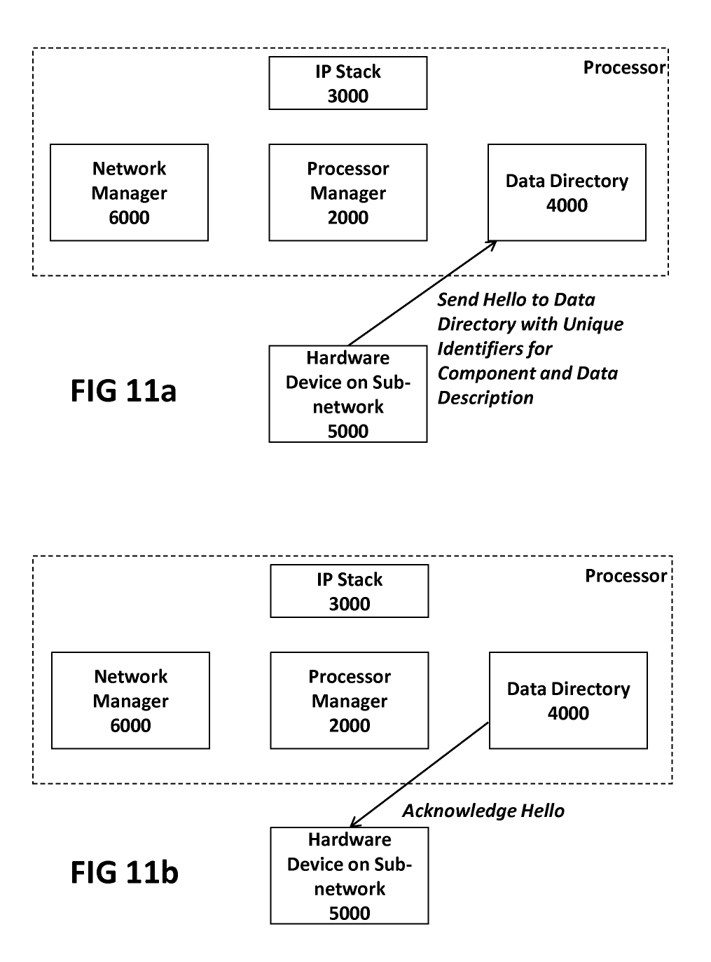

Once a node has received the Data Directory 4000 core component address message, it is obligated to send a ‘Hello’ message to all Data Directories 4000 that it has been made aware of. Sending of these messages, as illustrated in FIG. 11a, simply asserts the presence and identity of the device to the Data Directory 4000. Identity consists of a globally unique data value guaranteed by use of a standard such as the Microsoft GUID. The device address is carried within the message header as the “source” field in the “source”/“destination” address pair. Also included in the message is a unique identifier for the data description that defines the device's capabilities (to be described later).

Upon receipt of the ‘Hello’ message by the Data Directory 4000, the identity of the device is compared to a locally managed list of registered components. If the device presents an identifier not previously seen, it is added to a list of components to be registered. If it has previously registered, the case in a situation where the device resets or has been intentionally power cycled, the device is simply added back to the list as “re-activated”. These two cases are handled differently as will be described in paragraphs to follow. In either case, however, the Data Directory 4000 acknowledges receipt of the Hello message back to the device to inform it that it's assertion has been received (as shown in FIG. 11b). The Data Directory 4000 adds each asserted device to a queue of components that need to be registered. The addition to a queue allows for the management of the registration feature in large systems such that the activity can be strictly coordinated to avoid an unmanageable “flood” of registrations that cannot be handled.

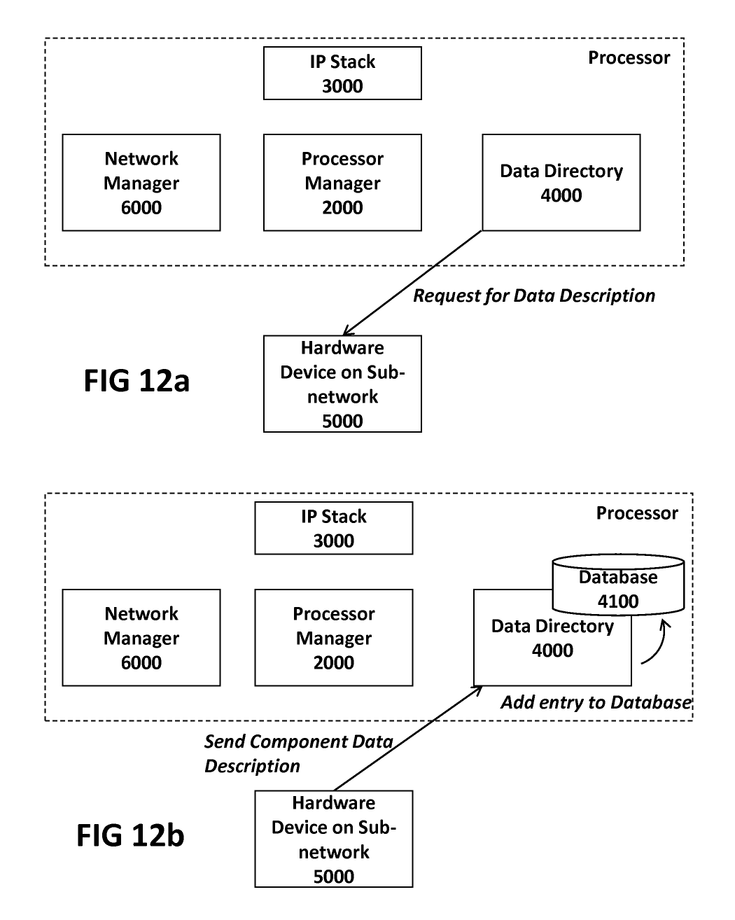

The Data Directory 4000 will continuously look to its registration queue and work off the list of components that are awaiting registration. Registration of a component is initiated by sending a message to that device requesting its data description as shown in FIG. 12a. The data description is an extensible mark-up language (XML) text string that defines the capabilities of the device.

When the device receives the message from the Data Directory 4000 requesting its data description, it complies by sending a Component Data Description message encapsulating the actual XML description text string. This may reach the Data Directory 4000 in one or many messages, depending on the size of the description. Upon complete receipt of the data description, the Data Directory 4000 parses (or interprets the contents of) the XML string, extracting the information necessary to understand the capabilities of the device, the message that it can consume or provide, the formats of the variables in those messages, and metadata and properties that augment the interpretation of the primary structural features of the data description. All of this information is added to a database of capability (as shown in FIG. 12b) that will be searchable by any “user” of the system (person, device or software application) to search for system data with specific characteristics.

Registration of Applications: An almost identical process is followed when registering an application with the Data Directory 4000 (as contrasted to registering a device resident on a network attached to a node).

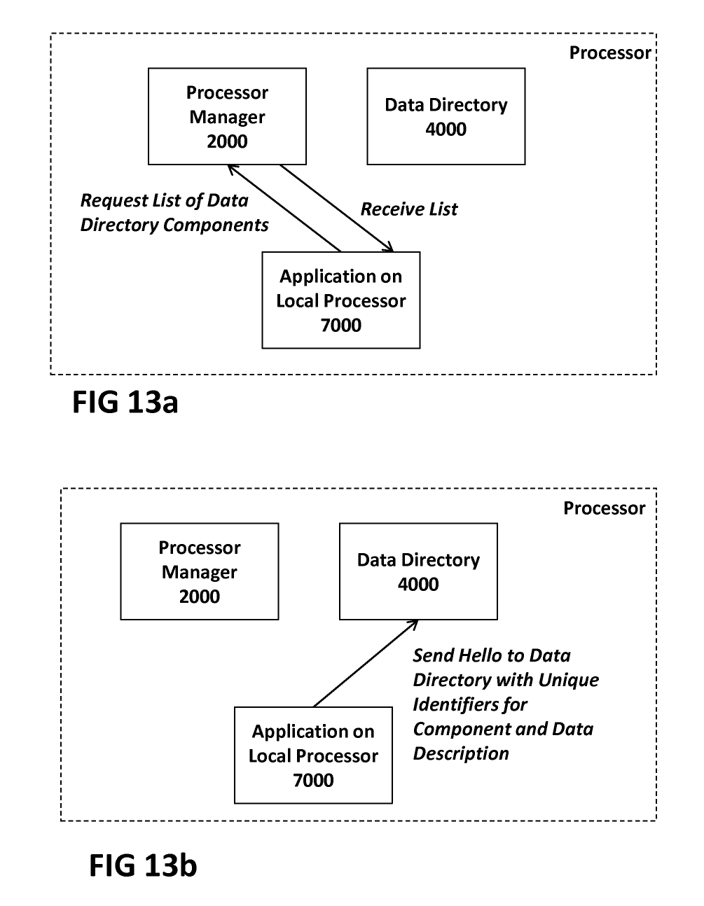

When an application is started on a processor, the application uses the well-known address of the Processor Manager 2000 to retrieve a list of Data Directories 4000 in the system (as shown in FIG. 13a).

For each Data Directory 4000 in the list obtained, the application will send the same ‘Hello’ message (shown in FIG. 13b), sent by devices, effectively expressing its identity and asked to be recognized for addition to the Data Directory's database 4100. Note that while the figure illustrates communication between an application and a Data Directory 4000 on the same processing node, they may be resident on separate processors—utilizing the network addressing mechanisms facilitated by the Network Managers 6000 to exchange messages over sub-network connections.

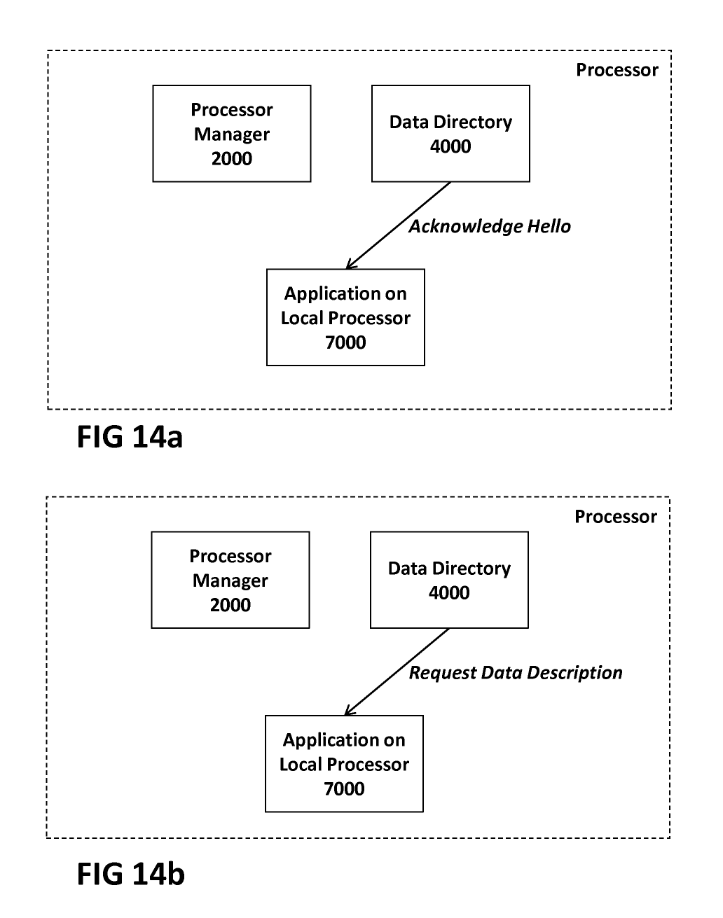

As with devices, the ‘Hello’ message is acknowledged by the Data Directory 4000, allowing the application to know that its request has been received. This interaction is depicted in FIG. 14a.

When the Data Directory 4000 is ready to receive the application's registration, a Request Data Description message is sent, alerting the application that it should initiate the registration process (as shown in FIG. 14b).

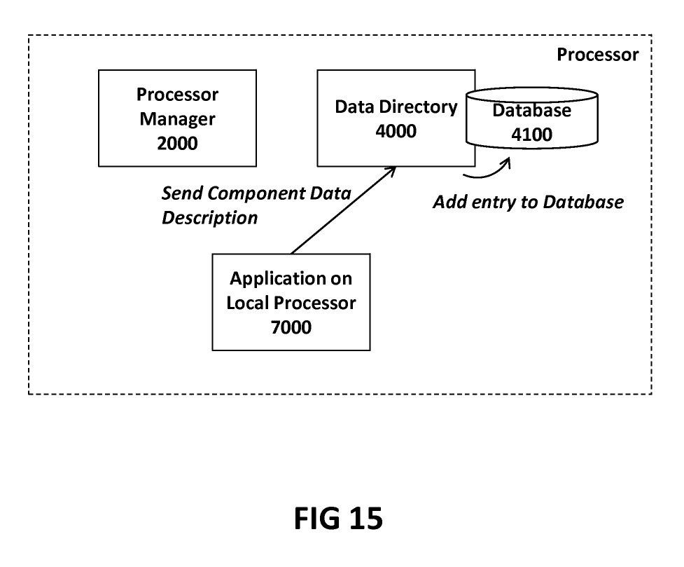

As in the case of devices, the application registers by delivering an XML data description in one or multiple messages to the Data Directory 4000. The data description conforms to the same format as that provided by devices—an important concept for the invention. An abstraction of capabilities is enforced such that the distinction between a device and an application is unimportant. Once again, that data description is added to the component Database 4100 as shown in FIG. 15.

Support of Rich Data Query: The features associated with registering components in the invention's data architecture have been described. The mechanisms for finding and attaching sources of data to consumers that require data of a specific prescription are described below.

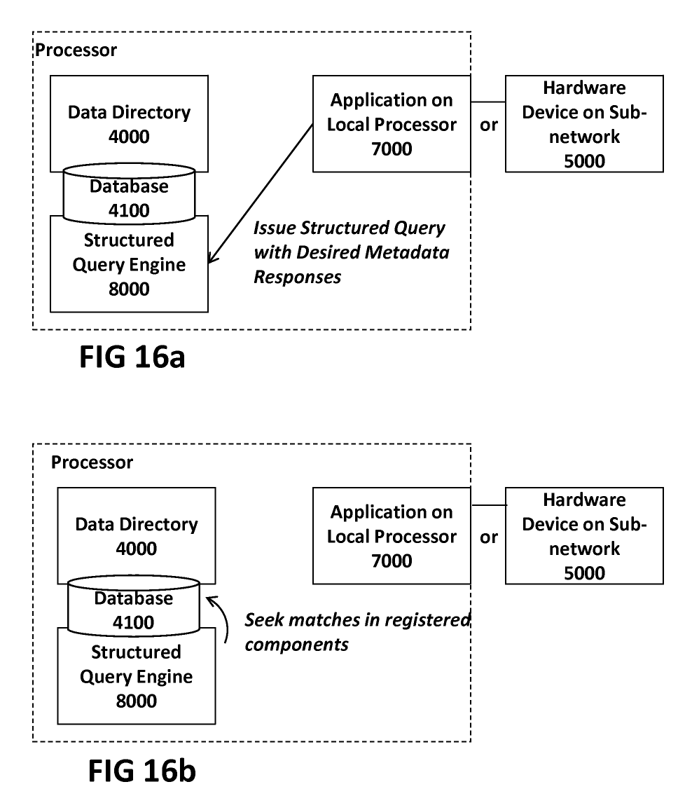

It was previously explained that the repository of information related to the data available within the system is contained within the Databases 4100 maintained by the Data Directories 4000. The Data Directory 4000 provides a data interface to a Structured Query Engine 8000 (shown in FIG. 16a) that can be accessed by any interested potential consumer of system data to find what it seeks. The query issued through the interface is an expression of the atomic “kind” of data desired (at the variable level) and a collection of optional metadata “qualifiers” that more richly describe the attributes to which that data should conform. Examples are: 1) the frequency at which the data can be delivered, 2) the accuracy of the data, 3) the frequency at which data can be taken, etc. Specification of these metadata “desires” can be exact (meaning that there must be an exact match to the conditions expressed) or conditional (such that any match greater than the value passed, for example, is of interested).

Accompanying the query itself is a list of desired metadata return values that can be used to better understand the matches that are returned. These need not be the same metadata qualifiers that were passed as the criteria for the query search. As an example, we may ask for sources of home or office air temperature data and augment that search by asking only for sources that can be provided on an hourly basis or more frequently. Along with this query specification we may also ask that when we receive back our list of matches we also get the name of the interface that it is encapsulated within—or the units that the value is expressed in. FIG. 16a shows this structured query for data being sent from an Application on Local Processor 7000 to the Structured Query Engine 8000.

Once the query specification has been received by the Data Directory's Query Engine 8000 (as shown in FIG. 16a), the engine will traverse the database looking for matches using the expression (depicted in FIG. 16b). The match is essentially the identifier of the message that contains the match data (including its address in the network, the identifier of the interface that the message is contained within, the identifier of the message itself, and the position of the variable within the message. This is sufficient information for an interested consumer to subscribe to the message, extract, and interpret the data.

Along with the list of actual matches is any query metadata that accompanied the request. Referring back to our example in the previous paragraphs, we would get back the source information about messages that contain the temperature change rate data that we seek, but would also get back the names of each interface that contains that rate data and the units that each of those rate variables is expressed in (which could be degrees per second, radians per second, revolutions per minute, etc). This information is all acquired by traversing the database using the primary query specification as the key and then pulling the requested metadata from the associated local structure as necessary.

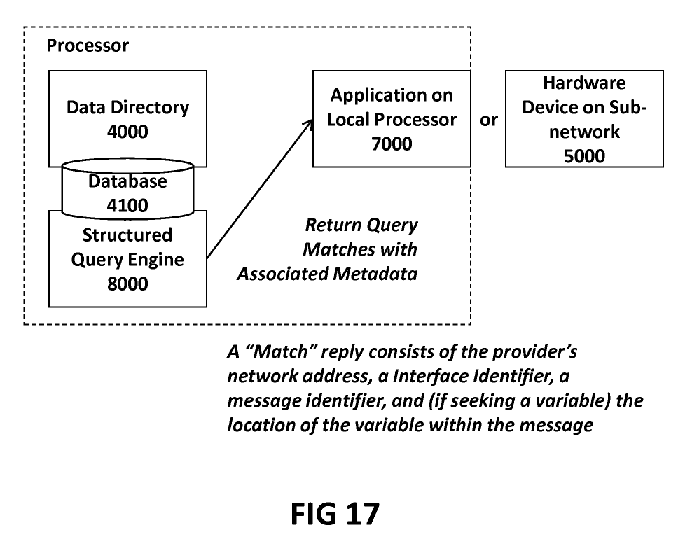

FIG. 17 depicts the results returned to the querying of an Application on Local Processor 7000 or a Hardware Device on Sub-Network 5000 from the Structured Query Engine 8000 after the matches and metadata have been retrieved from the database. The return is delivered in two messages. The first message includes the actual match information (address, interface identifier, message identifier, and the variable's location in message). The second message (which is returned only if optional descriptive metadata has been requested by the querying component) contains a two dimensional structure where the rows map to individual matches and the columns consist of one or more “label”-“value” pairs, where each “label” is the string name of the metadata tag that was specified as a desired return element, and the “value” is a string containing the value of the metadata itself The example previously cited mentions the return of units associated with the match. In this example the metadata label would be “Units” and the value might be “deg/sec”, both standard-expressed strings well know and defined in the common data dictionary.

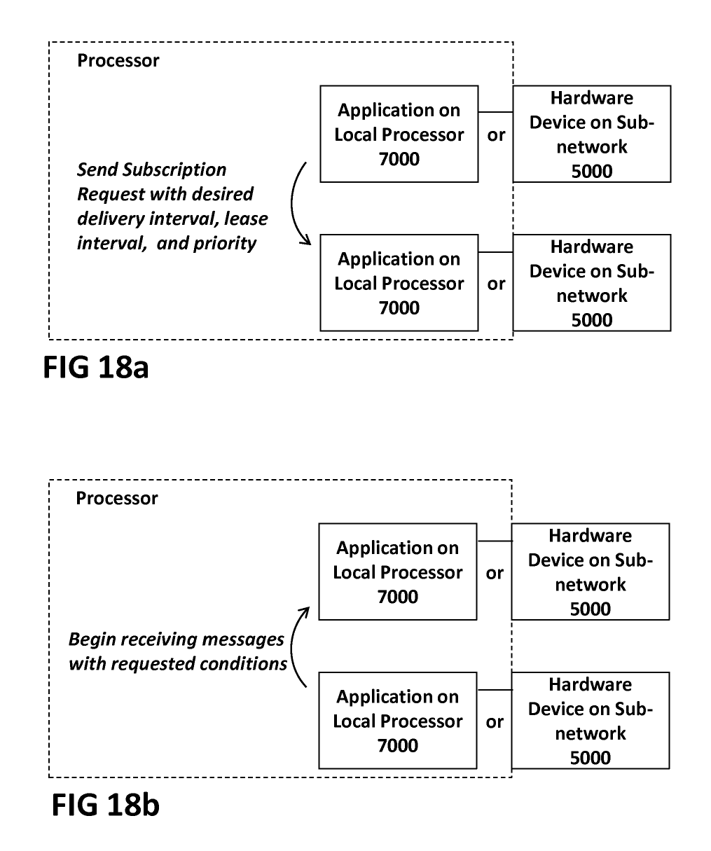

Data subscription management: Once the requesting component has received matches to its query, it is in a position to access the sources of data. This is accomplished by going directly to the provider (as shown in FIG. 18a). Recall that the address of the matching providers has been returned, along with the interface and message identifiers. To initiate delivery of messages from a provider, the consumer sends a subscription request. The invention provides the ability to request messages being sent at the native deliver frequency (defined in the provider's metadata) by default, but the consumer may optionally specify a divisor value in the subscription request to indicate to the provider that it only wishes to receive 1 of every N messages produced. Additionally, a lease period, or time frame that information is needed, is in the subscription request. Furthermore, the priority of the message may be specified. This allows mechanisms at the network layer to deliver higher-priority messages ahead of lower priority messages.

Once the provider has received the subscription request message, it begins sending the messages at the divisor rate and priority requested by the consumer (as shown in FIG. 18b). The provider obviously knows the rate of message production and will send the messages for lease period that has been specified in the subscription request. At the end of the lease period the provider will stop sending messages.

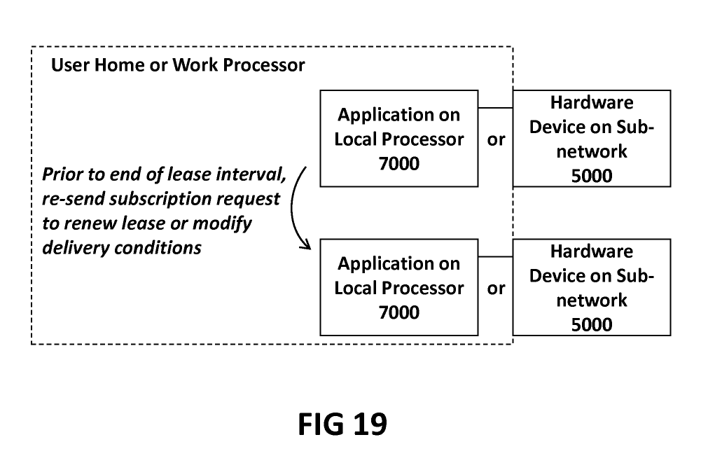

The lease mechanism is a way of ensuring that message relationships between peers in the system do not continue if consumers fail. The provider of data gives a “hint” of a reasonable lease period in its message metadata (as it is in the best position to make the recommendation). It is the burden of the consumer of a message to receive this value and send “lease renewals” (the same message used for a subscription request) before the lease period expires. This periodic renewal is shown in FIG. 19. If this relationship is honored (it is automatically honored if using the application programming interface of the invention), messages will be delivered between the provider and the consumer without interruption.

Data Centric Streams is a feature of the invention's application programming interface that provides an additional layer of abstraction to the software developer. While the rich query mechanisms described in previous sections exposes a powerful capability to find suitable sources of data within the system, it is still the burden of the consumer receiving the query matches to determine how to manage multiple matches.

Data Centric Streams Class 9000 is a software facility that provides a further abstraction that allows a data consumer to not just specify the type of data that it wants, but also specify the way that multiple sources of similar suitable data should be handled.

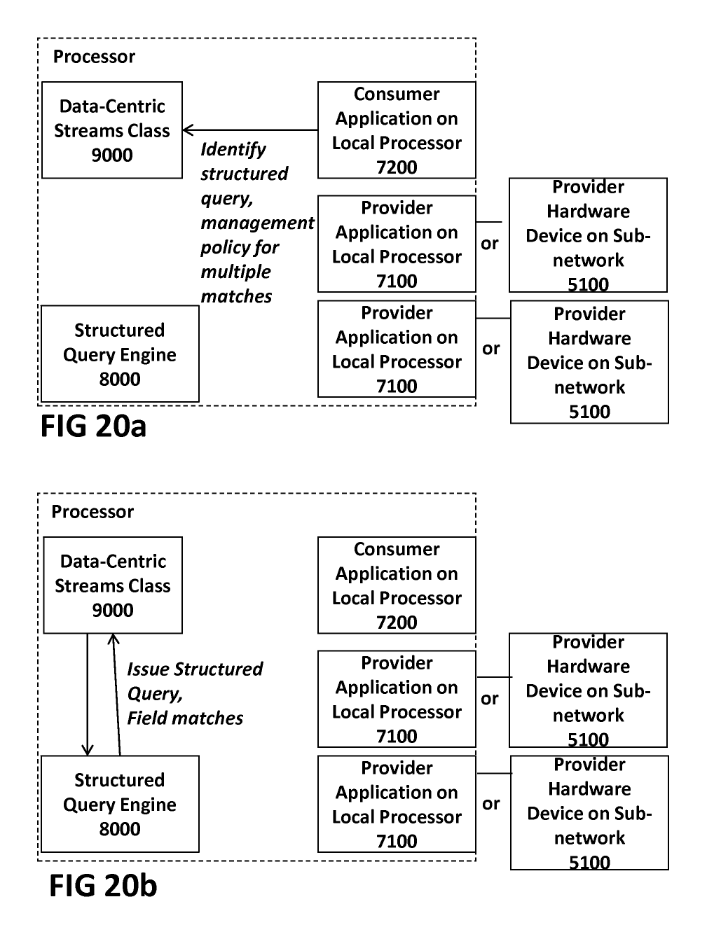

As in a pure rich query, the consumer specifies the query parameters and metadata, but additionally specifies the management policy for those data “streams”. Both of these are expressed through the Data Centric Streams Class 9000 application programming facility (interface) as shown in FIG. 20a.

The policy for management of multiple query matches includes minimally the following controls:

1. Specifying the criteria by which the “active” message stream should be selected. This is typically based upon a metadata qualifier such as “accuracy” or “delivery rate”. The consumer of data will logically want the source of data that is closest to ideal for its application. In the “single active source” mode, the data centric streams class will subscribe the source stream that is most desirable, receive the messages, and hand them up to the consumer application. If the most desirable stream ceases to be delivered, the data centric streams facility will immediately select the next best match, subscribing that message stream—thus providing uninterrupted access to suitable messages without any intervention on the part of the consumer application.

2. Specification that only a single source shall be used at any given time (using the same selection logic as mentioned in 1), but applying post-processing of the active data stream. This can be the use of a pre-defined algorithm (such as a moving average filter to smooth the data) or a user-specified function that can be applied to the data (a scaling function is an example).

3. Specifying that all streams will be used in combination to provide a single result that is passed up to the consumer component 7200. The obvious option is a simply average of all stream samples into a single value.

Following paragraphs describe the interactions between the function blocks involved in facilitating the Data Centric Streams feature of the invention.

FIG. 20b shows that the structured query expressed in the call to the Data Centric Streams Class 9000 is passed to the Data Directory's Structured Query Engine 8000—exactly as if a consumer component itself had issued the query.

Just as in the Rich Query case, the engine returns matches and metadata, but instead of being fielded by the consumer application is it received and processed by the Data Centric Streams Class 9000.

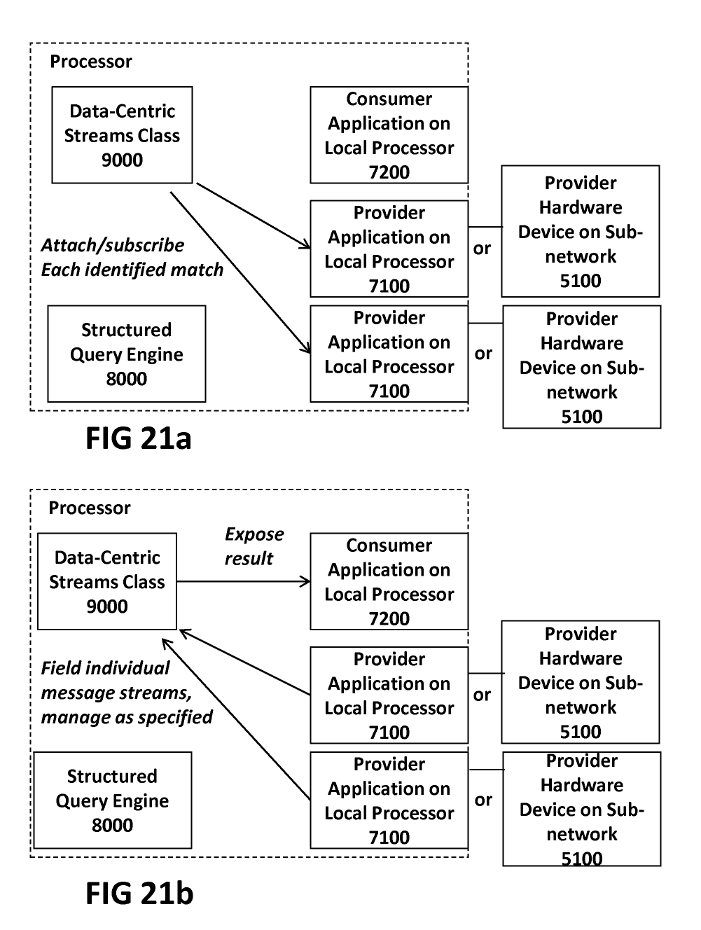

Based upon the stream management policy described in previous paragraphs, the Data Centric Streams Class 9000 will issue subscription requests according to need. Depending on the mode of operation, this may imply subscribing to just one provider's data stream or subscribing to all. FIG. 21a shows an example case where all sources are subscribed.

Subsequent to subscribing the data streams from providers, the providers will begin sending their individual message streams. Just as in a pure message subscription relationship between a provider and a consumer, the consumer is responsible for renewing leases with the provider to ensure that data continues to be delivered.

Based on the management policy specified, the Data Centric Streams Class 9000 will handle and process the messages, sending only a single stream to the Consumer Application on Local Processor 7200 using the Data Centric Streams Class 9000 (as shown in FIG. 21b).

The stream management policy may be changed at any time during execution. Based on the policy change this can result in a change in the active subscriptions with various matching providers or the way in which the messages received by the Data Centric Streams facility are processed.

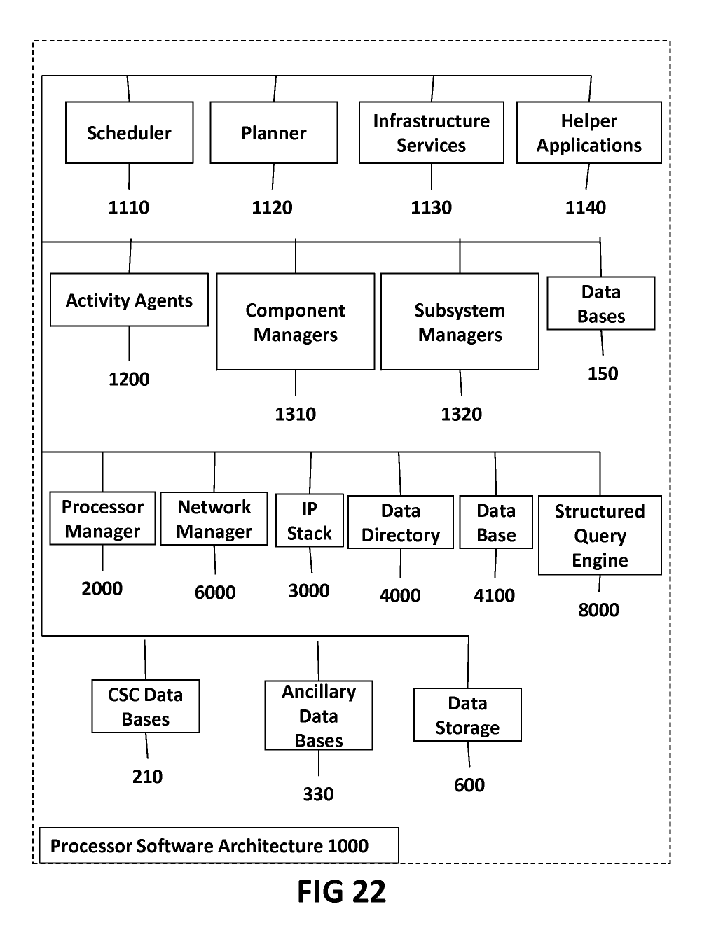

FIG. 22 depicts the architecture of software that will be hosted on processors depending on the actual implementation based on the user's needs and where the processor is located: in the user's environment, at the CSC, and at ancillary party locations. This is due to the different nature of activities at these locations. Additionally, the modules will be different based on different needs or functions of people at these locations. These are explained in more detail in subsequent figures.

In general, the Scheduler 1110 module is running the activities of the software and hardware per the needs of the location (user environment, CSC, or ancillary party location).

The Planner 1120 software is responsible for de-conflicting hardware, software, or human activities. It must work with the scheduler to perform its functions. This software module may not be required if there is not a need for operations that will run automatically.

The Infrastructure Services 1130 software modules perform various functions services such as the management and retrieval of data from libraries and buffer management.

Helper Application 1140 modules perform translation functions between hardware capabilities and the invention services to the user in the user's environment, the operator at the CSC, or an ancillary party.

Activity Agents 1200 are software modules that are specific to functional activities or initiating and terminating hardware operations. One example of an Activity Agent 1200 would be a software module that performed a check of the proper operation of a device such as the fall detection device and periodically sent a status message to the User's Main Processor 110.

Component Managers 1310 and Subsystem Managers 1320 are software modules that control hardware operations or collections of hardware operations that comprise a subsystem.

Data Bases and Data Storage 150, 210, 330, 600 are collections of reference information used by the other modules within the architecture (as previously described).

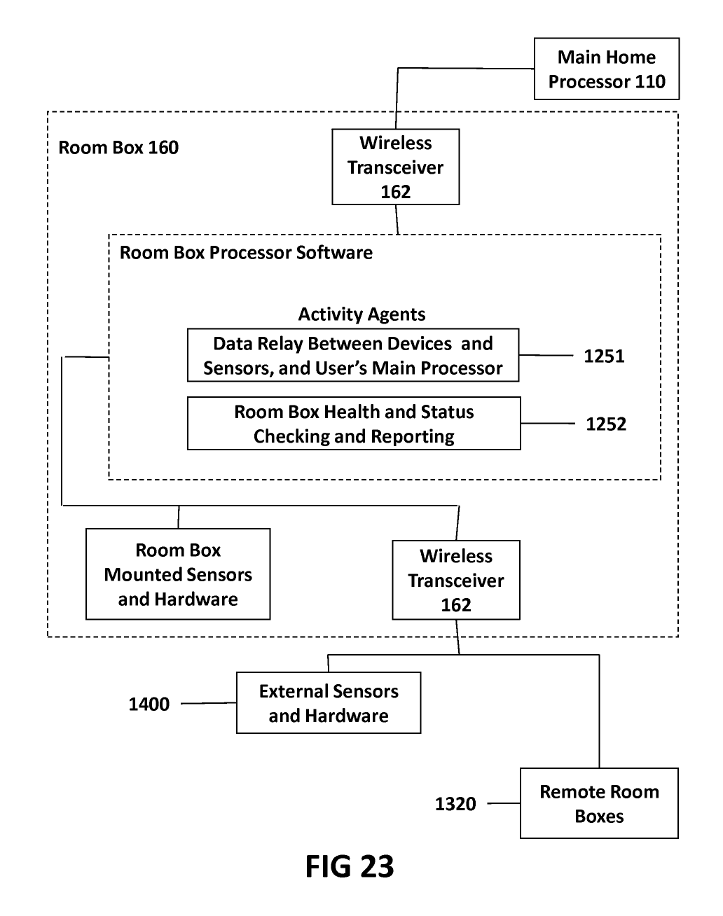

FIG. 23 depicts the software architecture in the Room Box 160. The Room Box Processor 161 hosts an RF Data Relay Between Devices and Sensors 1251, and User's Main Processor Activity Agent 1201 that either moves data from the Wireless Transceiver 162 connecting subordinate elements of the system (such as more Remote Room Boxes 1320 in the system or other hardware such as the Fall Detection and Help Request Device 800) to the Wireless Transceiver 162 that connects the Room Box 160 to the User's Main Processor 110. This RF Data Relay activity agent 1251 also moves the data coming from the sensors and hardware mounted on the Room Box 160 such as the battery voltage sensor or the smoke sensor.

The Room Box's Processor 161 also hosts a Room Box Health and Status Checking and Reporting Activity Agent 1252 that collects Room Box 160 component status information and sends it to the data relay activity agent for wireless transmission to the User's Main Processor 110.

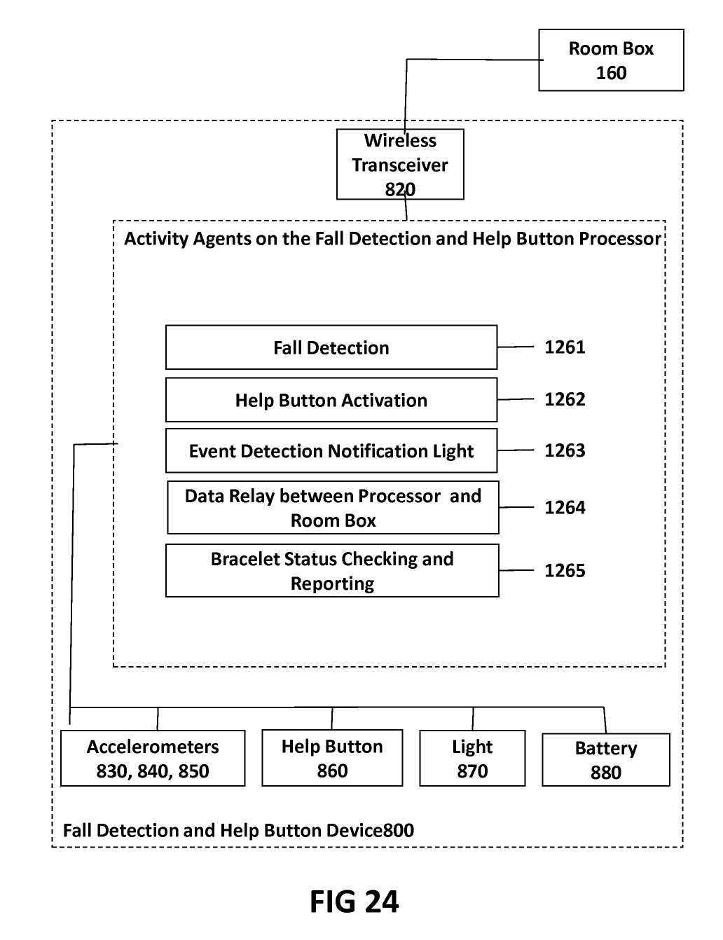

FIG. 24 depicts the software activity agents hosted on the Fall Detection and Help Button Device's Processor 810. The Fall Detection Activity Agent 1261 contain the logic to sense and report a fall to the User's Main Processor 110. The Help Button Activation Activity Agent 1262 senses when the help button is pressed and sends a corresponding data message to the User's Main Processor 110. The Emergent Event Detection Notification Light Activity Agent 163 senses when either the Fall Detection Activity Agent 1261 or the Help Button Activation Activity Agent 1262 is sending a data message and illuminates a light in the Fall Detection and Help Button Device 800 to let the wearer know that the system has sensed an emergent event. The data relay between the processor and Room Box activity agent 1264 controls messaging from the Fall Detection and Help Button Bracelet Device 810 and the Fall Detection and Help Button Device's Transceiver 820. The Device Status Checking and Reporting Activity Agent 1265 sends the device health and status data messages to the User's Main Processor 110 through the Room Box 160.

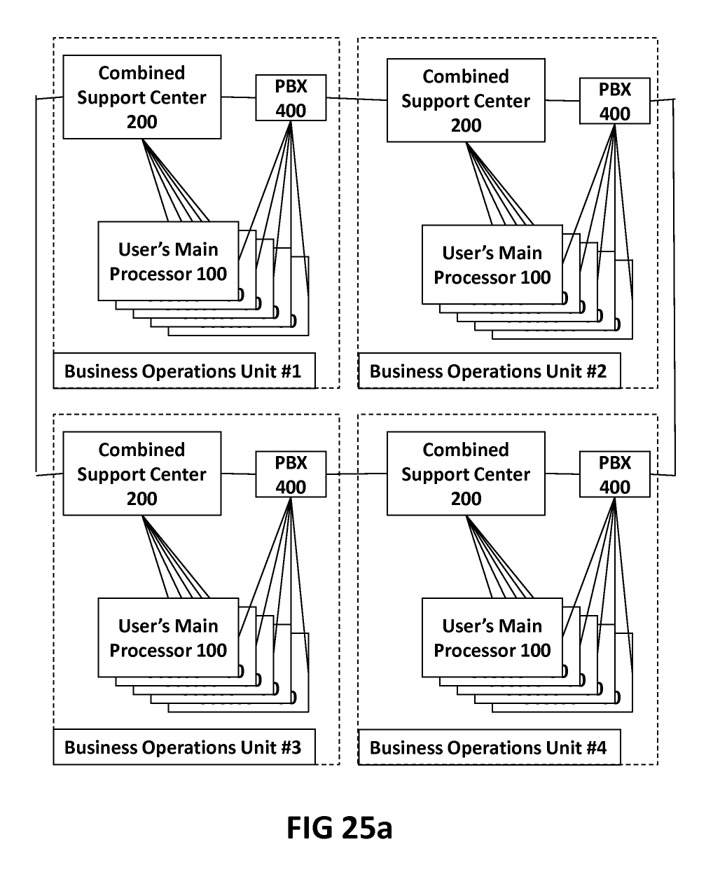

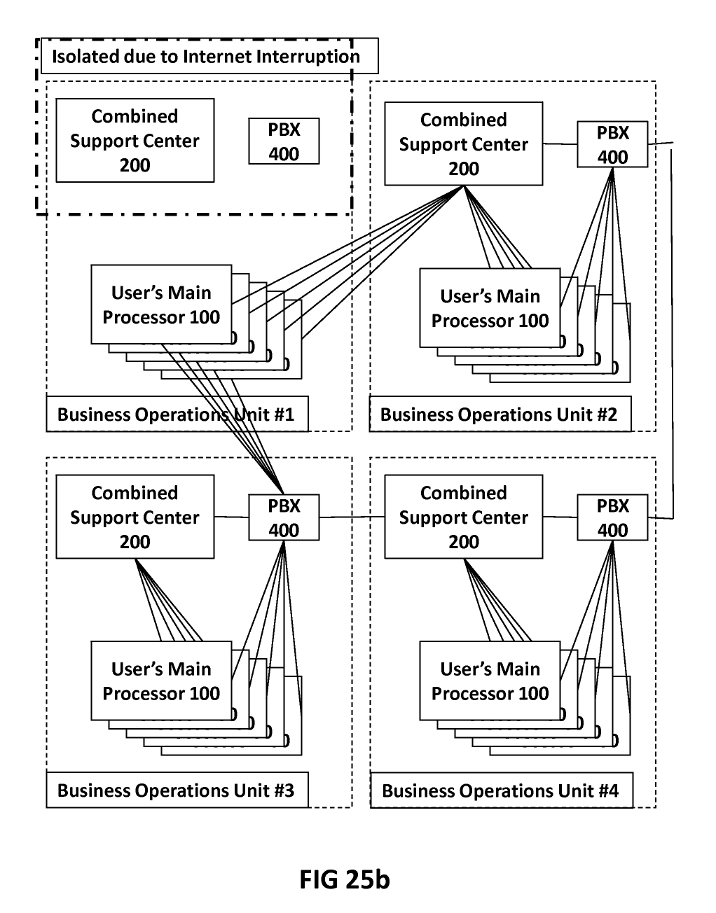

FIG. 25a depicts the nominal topology of a system of systems. The system is designed to set up Business Operations Units in which CSCs are assigned clients (the user's main processors), client data bases, back-up data bases for contingency support to other Business Operations Units, and PBX servers. The client assignment is made when a business unit is created, and is based on staffing considerations versus clients being served, and bandwidth availability.

FIG. 25b depicts how the topology can change to adapt to connectivity failures between User's Main Processors 110, CSCs 210, and PBX Servers 400. Within the CSCs and User's Main Processors 210, 110 are software modules that track the status and availability of CSCs and PBXs within the system of systems. In the event of internet or connectivity failure, the User's Main Processors 110 will temporarily join another Business Operations Unit, i.e., the client base of another CSC best able to handle the additional clients and the PBX best able to support additional User's Main Processors 110 that it supports. FIG. 25b depicts a situation where the CSC and PBX of Business Operations Unit #1 have internet connectivity failure. The CSC's clients automatically join the client base of the CSC in Business Operations Cell #2 and the PBX server of Business Operations Cell #3.

Creating software and hardware devices: People wanting to create new, ASPIRE-compliant hardware to integrate with the invention will need to obtain the ASPIRE Common Data Dictionary, the ASPIRE software schema (which defines the XML format of messages), and the ASPIRE libraries of Application Programming Interfaces (API). In this case, the APIs allow hardware developers to know how to describe their data (what is needed or produced) in an invention-compliant manner

Similarly, software writers or application inventors who desire to write software to use hardware or data from existing applications in different ways will also need to obtain the ASPIRE Common Data Dictionary, the ASPIRE software schema (which defines the XML format of messages), and the ASPIRE libraries of Application Programming Interfaces (API). As above, APIs allow application writers to know how to describe their data (what is needed or produced) in an invention-compliant manner

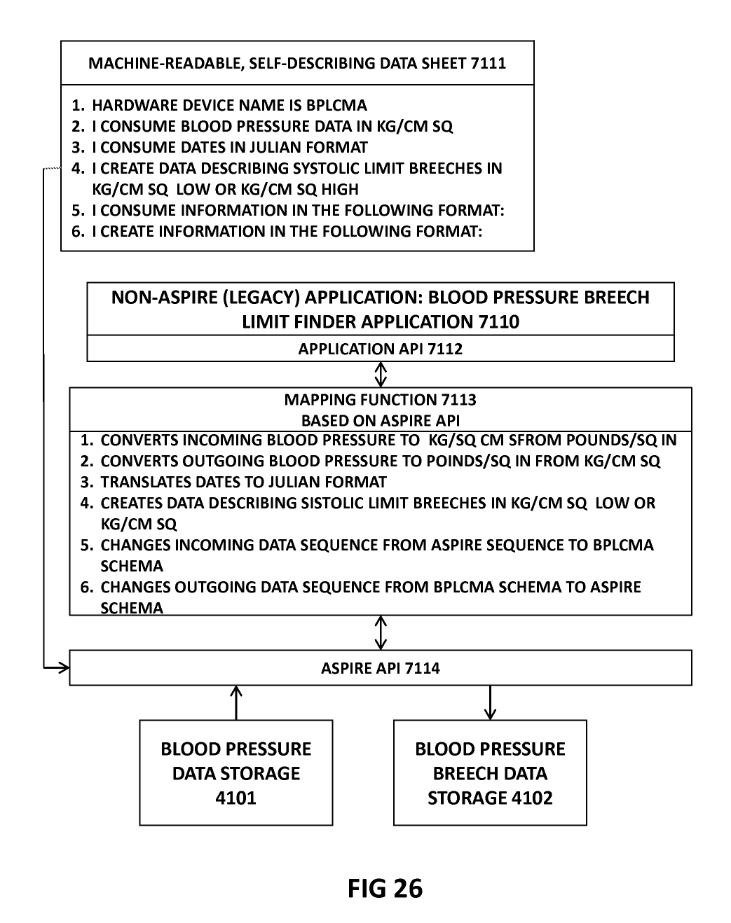

The creators of this invention intend to write software applications to be used in this invention. However, it is also the intent of the inventors to be able to leverage the creative talents of marketplace and be able to integrate existing and new non-ASPIRE compliant software applications that improve this invention's ability to support safe, independent living. FIG. 26 depicts an example of the architecture of a non-ASPIRE (legacy) compliant Blood Pressure Breech Limit Finder Application 7110 that uses information from a Blood Pressure Data Storage 4101 location and provides breech information to a Blood Pressure Breech Data Storage 4102 location. The two steps to integrate the non-ASPIRE compliant Blood Pressure Breech Limit Finder Application 7110 are as follows:

The first step is to write the Machine-readable, Self-describing Data Sheet (MSDS) 7111 that describes the data interfaces to the non-ASPIRE compliant software application. The use of the MSDS in the software device identifying itself to the system is described in the prior text referring to FIG. 8. The second step is to use the ASPIRE Application Programming Interface 7114 to create the Mapping Function 7113 that does the bridging between the ASPIRE-compliant components on the network and the non-ASPIRE compliant Blood Pressure Breech Limit Finder Application 7110.

### How to Use the Invention

This section will discuss the steps that a user of the invention would follow to put it into use and how the invention would be used on a continuing basis. The use is focused on the preferred embodiment of supporting independent living and increasing the quality of life of the user. The discussion below starts with identification of services required. This lets a user then decide on where in the home or work place to locate hardware and what software algorithms to host on what processors. The user can then decide on the topology of the invention. Discussed separately is how to set up and use the software standards and middleware.

The first step in using this invention is for users of the invention to decide on the services that they would like to provide to the home resident or work place occupant. Services categories and specific examples that can be created by this invention are listed below.

Time critical emergent events (i.e., events that require time critical responses) services include detection and notification of falls; physiological events such as heart attacks, breathing problems and diabetic problems; smoke; fire; carbon monoxide; extreme temperatures due to door or window being left open or air conditioner failure; break-ins; and the ability to summon help when an unanticipated emergency event occurs.

Physiological state and physical health monitoring, trending, and coaching include the following services: monitoring blood sugar levels, monitoring blood oxygen levels, monitoring weight and body fluid electrical resistance, monitoring blood pressure, monitoring pulse rate, reminding and monitoring medication consumption, reminding and monitoring diet, and reminding and monitoring exercise regimens to include physical therapy.

Facility and environment monitoring services include monitoring air temperature, air quality, humidity, lighting, and utilities. Control can be automatic or manual, from an in-home or in work place control device, or remotely.

Mental health and social services include providing quality point-to-point and multi-party video teleconferencing with professionals, family, friends; access to calendars and event notification; access to on-line media, books, periodicals, and games.

Quality of life and labor augmentation include the ability to integrate software applications and hardware devices into the home or work place that can perform physical or mental activities that a person would prefer not to do if that person was confident that it would still be done to satisfaction. This could include the use of robotic devices. Similarly, productivity services include the physical or mental activities that make a person, device, or process in the home or work place more efficient.

In addition to deciding what services are desired or needed, the invention user must decide where the services are physically needed in the home or work place. This will help the user determine the individualized hardware and software architecture to be created in the home or work place.

The preferred embodiment of this invention, services for monitoring emergent events, the physical facility, and environment are in all locations of the home and work place. The exception is that in the preferred embodiment of this invention, both the fall detection device and the device to immediately summon help are worn or carried on the user. These devices are integrated into a single device, the Fall Detection and Help Button 800, that sends a fall detection signal or ‘help’ signal by wireless link from the Wireless Transceiver 820 to a Wireless Transceiver 162 and Processor 161 in the Room Box 160 that retransmits the signal to a receiver at the Main

Home Processor 110.

In the preferred embodiment of this invention, Smoke Detectors and Carbon Monoxide Detectors 165 are located in the Room Boxes 160. To ensure ubiquitous monitoring and services, Room Boxes 160 are located in all major rooms. They can also be located in outdoor areas such as yards and out buildings. As required, the Room Boxes 160 can act as relays between the main home outlaying Room Boxes 160. In the instance that the output data from these detectors are not in a compliant format, the signal will be converted either at the Room Box processor 161 or sent direct through the Room Box's Wireless Transceiver 162 to the User's Main Processor 110. There, the software device or algorithm associated with smoke and carbon monoxide response will initiate a voice message to the user indicating the condition and recommending the user leave the home or work place. Simultaneously, the User's Main Processor 110 will send a signal indicating that smoke or carbon monoxide detection to the Combined Support Center's Processor 210. At the CSC 200, when smoke or carbon monoxide is detected in a home or work place, the CSC operator will initiate a video call to the residence. The CSC operator then follows the escalation procedures established for that user.

Other monitoring devices such as door and window position sensors, room temperature and humidity sensors and controls, utility sensors and controls, may or may not be integrated with the Room Boxes 160. Quality of life and labor augmentation devices will probably be separate from the Room Boxes 160. If separate from the Room Box 160, the device will have a wireless transmitter to send its data to and receive data from the Room Box 160. The Room Box 160 will either process the data on a processor built into the Room Box 160 or it will relay the data to the User's Main Processor 110 where software applications will use the data to provide the service needed by the invention's user.

Because the Room Boxes 160 are critical to relaying information from devices monitoring for emergent events, they will have a battery back-up to enable functioning for limited periods of time during commercial power outages. Additionally, the Fall Detection and Help Button Device 800, and Room Boxes 160 will generate status signals sent to the main home or work place processor that will be forwarded to the CSC for its operators' awareness of problems with user monitoring capabilities. Similarly, the CSC will send status signals to the User's Main Processors 110 to keep users informed when there is a loss of that support function.

Invention topology: Another critical first step is the implementation of this invention is the development of the topology. In the preferred embodiment of this invention, owners and operators of a home and work place monitoring and control system will have in mind some number of user home or work environments 100 that they desire to support and what kind of support they will provide. This will determine the number of Combined Support Centers 200 they will set up. For home health and safety monitoring, the limit to the number of clients will be that number that has minimal waiting time before an attendant can answer the call or respond to a time critical event. Similarly, the PBX server 400 will be sized to support the anticipated number of ‘calls’ and location will be primarily determined by connectivity considerations such as bandwidth, reliability, etc.

Use of the Invention in the Home Environment 100: The preferred implementation of the invention is supporting a user in the home. The Main Home Processor 110 is selected so that its memory size and processing capabilities will run the software needed to support the client.

The resident or client in a home or work place interfaces to the system through a Monitor 120 and Audio System 130. For elderly residents, the Monitor 120 will be large enough to be readable from across the room and choices must be simple enough to be unambiguous. For clients that only want basic safety and health monitoring support from the system, the monitor 120 will be touch sensitive so that clients do not have to use a keyboard and mouse. For residents that want to take advantage of all the capabilities of computers, they will be able to use a mouse and keyboard to interface with their User's Main Processor 110. Residents will have the option of what screen saver they desire. One choice will be a display of a daily calendar and event reminder that will have a check-off feature so that residents can keep track of what they have accomplished, such as exercising, dieting, or medicating, and this information can be recorded in data files accessible by a CSC 200, and Ancillary Parties 300 such as medical professionals, relatives and friends, and coaches for diet/medication/exercise monitoring and compliance. In the case of a medication reminder, clients will have the choice of having images of the actual pill or the image of the bottle of liquid with dosing instructions displayed, “Take 2 tablespoons with 1 glass of water.” Another choice will be to not use a screen saver but display the main video conferencing page which will display a ‘Help’ button, and other buttons that will initiate video calls to parties that they have designated such as medical providers, friends, or relatives. One option will be a button that opens a phone book to allow initiation of calls to other parties. Users will have the ability to video record events such as medicating.

The processor will host speech generation software that will vocalize calendar events, warnings to the resident based on what is sensed in the environment, and other announcements as programmed by the User's Main Processor 110 or coming from the CSC 200. The User's Main Processor 110 will have the option of activating speech recognition capabilities. This is an option because the reliability of speech recognition is affected by ambient noise from television and radio, from external noise such as street noise, the acoustics of a particular room, and the acoustic qualities of a person's speech, and the user's hearing ability.

The User's Main Processor 110 will host a video teleconferencing capability. The teleconferencing call can be initiated by the resident by touching the User Video Screen Interface 120 or voice commanding when speech recognition is activated. The Video Camera 140 will be high definition and can be either integrated with the monitor or can be detachable. The monitor will support the resident being able to recognize facial expressions and skin tones of the person to whom he is talking. The resident's camera will enable the same for recipients of the resident's video picture. The camera will be used to help the CSC 200 personnel and medical providers evaluate the physical condition of the resident. Detachable cameras can be positioned so as to show cuts or bruises following a fall. This will allow CSC 200 personnel to make better informed decisions on whether a person may need time-critical medical attention, needs to video teleconference with medical personnel, or has does not have a time-critical or medical issue, but needs advice. One feature of the video conferencing is the capability to connect three parties in a video call. Each person sees and hears the other two. This capability increases the effectiveness of the system to provide security and safety to clients. For example, two doctors specializing in different fields could be simultaneously talking to a client (with conditions in those two fields) to hear the client symptoms and discuss how to best respond to a patients problems. A second example is a doctor, a part-time home care assistant, and a client could be simultaneously connected. The doctor could hear the symptoms from the client, the part time care assistant could provide background information and history, and the doctor could direct what the patient should do and what actions the home care assistant should take that could include changing the care plan.

To provide pervasive support throughout the house or workplace 100, a home or workplace should be equipped with a Room Box 160 in every room. Additionally, Room Boxes 160 will be located in the yard, garages, and detached spaces such as workshops, green houses, etc., to ensure monitoring the client and environment where client may be spending significant amounts of time. It is not envisioned that Room Boxes 160 will be in closets or pantries but they are not precluded when a closet or pantry has a device or sensor that needs to be interfaced to the User's Main Processor 110. The Room Box 160 will relay both two-way audio as well as send data between sensors in the room and the User's Main Processor 110. Additionally, the Room Box 160 can be set up as a two-way relay between an outlaying Room Box 160 and the User's Main Processor 110.

Individual sensors, monitoring, and living assistance devices; their use and integration into the home and workplace:

One intention of this invention is to facilitate rapid and synergistic development and evolution of hardware and software by engineers and inventors that have no connection with each other. This is accomplished by the use of the hardware, software, and data interface standards that this invention specifies. Strict adherence to these standards will allow hardware developers and software writers to create a hardware capability or software module, connect to the network or be loaded onto a processor, and the hardware or software item will automatically integrate itself and start to perform its intended function. The best mode for use of this invention is to create a baseline of capabilities in a field, such as home or workplace monitoring and assistance, and let hardware inventors and software developers improve on what is initially fielded. The following paragraphs describe the initial hardware and software devices to be fielded and describe how they can be improved. A synergistic and spiral manner is seen as the best way to rapidly advance the state of the art and technology in both the field of this invention and the best mode of its use.

Addressing Emergency Events:

The time critical emergent events addressed by this invention are detecting and responding to falls, home and workplace fires and smoke, dangerous carbon monoxide levels, and other situations that the resident desires immediate help to address.

Fall Detection: This invention includes the integration of a fall detection device 800 that is worn by a user in a bracelet. Accelerometers 830, 840, 850 in the device sense motions and orientations that are pattern-matched to a motion-position library in the memory of the device. When a match occurs, the fall detector sends a signal by wireless to the Room Box 160 which relays the signal to the User's Main Processor 110 which is hosting software to monitor sensors within the home and workplace system. The Fall Detector and Help Button Device 800 also illuminates an indicator light 870 in the bracelet indicating that it is sending out an emergency signal to the Room Box 160. When that software module in the User's Main Processor 110 receives information that the Fall Detector and Help Button Device 800 has a fall indication, it initiates activities to corroborate the fall. It queries the Fall Detector and Help Button Device Processor 810 for indications of subsequent motion indicating that the person had gotten back up and is moving. The User's Main Processor 110 simultaneously looks at indications from the Room Box-mounted infra-red (IR) detector 164 to identify motion in the room indicating the person is back on his or her feet and is moving around the room. Following a fall detection indication from the Fall Detector and Help Button Device 800, if there is motion detected by the bracelet-mounted fall detector 800 or the Room Box-mounted IR sensor 164, the User's Main Processor 110 stops the response procedures and monitoring for emergent events. However, if there is no detected motion from the Fall Detector and Help Button Device 800 or motion detected by the room-box IR detector 164, the fall detection software module in the User's Main Processor 110 will initiate generation of speech, through all Room Box-mounted speakers 163 in the house, asking the resident, “Do you want me to call for help?” The verbal query is generated from all Room Box speakers to alert any other people in the home that an emergent event has occurred so that they can respond to the fall victim. If there is no answer, or if the person's voice is recognized as saying, “Yes” or “Help,” the User's Main Processor 110 will send a message to the CSC Main Processor 210 indicating that the resident has fallen. When the User's Main Processor 110 is generating the verbal query, “Do you want me to call for help,” the monitor will also display that question with a “Yes” and “No” button shown. The resident can push the “No” button and stop the call. This will also reset the software to be ‘listening’ for the next fall event. Within the existing state of the fall detection art, scores of algorithms exist to identify fall events. The use of other home sensors to corroborate the fall event increases the accuracy of the detection. It is anticipated the future inventors and software writers will conceive of other devices that can more reliably detect or corroborate falls. If their hardware and software inventions are written using ASPIRE software schema and they use ASPIRE Application Programming Interfaces, they will be able to immediately be integrated into the home system and used without having to write software to adapt the specific device to the home system. At the CSC 200, when a fall detection indication is received, the CSC operator initiates a video call to the residence. The CSC operator then follows the escalation procedures established for that user.

Help Button: In addition to hosting the fall detection device, this invention's bracelet also hosts a ‘Help’ button 860. When the button is pressed by the person wearing the bracelet, the electronics in the bracelet wirelessly sends a signal to the Room Box 160, which relays it to the User's Main Processor 110. There, software that is monitoring for this signal initiates generation of speech, through all Room Box Audio Interfaces 163 in the house, asking the resident, “Do you want me to call for help?” If there is no answer, or if the person's voice is recognized as saying, “Yes” or “Help,” the User's Main Processor 110 will send a message to the CSC's Main Processor 210 indicating that the resident is requesting help. At the CSC 200, ‘Help’ signal, or user's verbal call for help is received, the CSC operator initiates a video call to the residence. The CSC operator then follows the escalation procedures established for that user.

Smoke and Fire Detection: Each Room Box 160 hosts a smoke detector 165. When smoke is detected, the Room Box 160 sends a signal to the User's Main Processor 110. There, the Emergency Detection and Reaction software is monitoring for this signal and initiates generation of speech through all Room Box-mounted speakers 163 in the house stating “Warning, fire.” Additionally, the warning is displayed on the User's Main Processor 110 Video Screen 120. Simultaneously, the Main Home Processor 110 sends information to the CSC Main Processor 210 indicating a fire and the Room Box 160 where the indication occurred. The CSC operator then follows the escalation procedures established for that user.

Carbon Monoxide Detection: Each Room Box 160 hosts a Carbon Monoxide Detector 165. When carbon monoxide is detected, the Room Box 160 sends a signal to the Main Home Processor 110. There, software is monitoring for this signal and initiates generation of speech through all Room Box Audio Interfaces 163 in the house stating “Warning, carbon monoxide danger.” Additionally, the warning is displayed on the Main Home Processor 110 display monitor. Simultaneously, the User's Main Processor 110 sends information to the CSC Main Processor 210 indicating a dangerous level of carbon monoxide and the Room Box 160 where the indication occurred. The CSC operator then follows the escalation procedures established for that user.

As with fall detection, there are within the existing state of the smoke and carbon monoxide detection art, scores of existing devices. However, as new devices are created that increase reliably of smoke and carbon monoxide detection, those that are created to be compliant with our inventions standards will be able to be immediately integrated into the Room Boxes 160 and the home system without having to write software to adapt the specific device to the home system.

To increase CSC operator situation awareness of the severity of emergent conditions in a home or work place, the User's Main Processor 110 will tailor the message that is sent to the CSC main processor to indicate when more than one emergent event is happening simultaneously in a home or work place and what the events are. The purpose is to allow the CSC Main Processor 210 to increase the priority of the response over less time critical situations.

Physiological Data Monitoring: The current art of physiological data measurement includes scores of devices that can connect to home computers for home analysis or can move the data by internet to remote sites for storage, analysis, or subsequent reference. The devices include but are not limited to the following: blood glucose meters to determine blood sugar levels, blood oxygen meters to measure oxygen levels in blood and can also measure heart beat rate, blood pressure cuffs that measure blood pressure and also measure heart beat rate, scales that measure weight and wirelessly send it to a home computer, as devices that use electrical resistance to measure body fat and other indicators of potential heart problems. There are numerous diseases or conditions that are addressable by this invention. One is diabetes. In the preferred mode of this invention, a resident will use a commercially available device that analyzes blood, produces a blood sugar level reading, and records the level in a stand-alone computer 600 that displays readings. Many of these can be directly connected to a laptop or desktop computer, or connected by relay through a Room Box 160, and the readings transferred to files saved in that computer or on a storage location in the network. In this invention's preferred mode, that computer is the User's Main Computer 110. This invention will use software that will retrieve the data files, and perform assessments and analysis as needed by the user. This could range from simple identification of a value generated by the physiological measuring device, to trending, to establishing relationships with other items of data from other monitoring devices on the network. This information would be used by medical professionals, friends, family members, service providers, and coaches. This software will be written to conform to the standards which will give it several capabilities. The software can be hosted on any computer on the network, and it can find data of any client that is also on the network. When written to the software standards, the software will not have to have additional software to adapt it to the host computer.

This invention is intended to enable new hardware and software capabilities that are specification compliant, to easily integrate into the system and immediately function without adapters. For those hardware and software capabilities that are not specification and standards compliant, hardware and software ASIMs will be used to facilitate their integration.

Living Assistance, Care Plan Compliance Assistance and Monitoring: In its best mode, this invention assists people with normal daily living activities, with actions recommended by medical professionals, and with medicating; and it can assist the monitoring of these by professionals, care givers, and friends.

Software will be hosted on the User's Main Processor 110, the CSC Main Processor 210, or any other location on the network that will be able to create daily calendars presented visually on the monitor or verbalized to remind people of when to perform certain hygiene functions, when they should take certain medicines, when and what they should eat and should not eat, what appointments they may have, what clothes they should wear, when to perform certain household chores such as taking out the garbage, feeding pets, changing litter boxes, etc, and when to exercise. The basic method of compliance verification will be through the use of checklists that will be displayed on the computer monitor that the home resident checks off. For visually impaired, these will be verbalized and when the acoustic environment permits, answers from the home resident will be spoken. In the case of home residents that cannot reliably respond to checklists; medical professionals, care givers, friends, and relatives can make a video call to the home resident and watch the person take medication at the right time and in the right dosage; watch a person eat specific foods; and watch a person perform exercises. Software that senses movement in rooms will be used to track movement through a house to confirm that a person who was directed to walk a certain distance every day, does walk that distance. The current art of body movement recognition software exists to support computer gaming. This will be adapted to enable monitoring and coaching of home residents who have prescribed physical therapy exercises to comply with.

The invention in its best mode will also be used to verify a person's medical conditions and status by certain stake holders of both the well being of a person and the quality of care being given that person. These stakeholders include family, hospitals, medical providers, and even insurance companies. Software programs will check on exercise, medicine, or diet compliance. Other software programs will evaluate physiological data to determine the continued existence or re-appearance of conditions that were thought to have been treated or cured. Some software programs will be used to confirm that services were rendered to users in their homes and other programs will monitor homebound people to confirm that they are truly homebound. These programs will be used by medical providers, insurance companies, and Medicare, and Medicaid representatives.

Home and Work Place Environment and Device Monitoring and Control: The invention in its best mode will also be used to monitor a person's environment and control devices in that environment. The current art of mechanical monitoring devices allows detection of the position of anything moveable in a home or workplace. Examples include exterior, interior, cabinet, and refrigerator doors; windows; and drawers. Similarly, the current art of temperature and humidity sensing allows monitoring of the temperature of rooms, devices, and detection of humidity in locations in homes and work places. Software programs hosted on the User's Main Processor 110 will look for values from these sensors and respond as designed which can include notifying the home occupant or the CSC 200, or actively adjusting the thermostat or humidity control. Logic will be included to look for potential overflowing toilets, bathtubs, open windows in rain storms, etc. As previously mentioned, this software will be hosted on the User's Main Processor 110 but could be hosted on any processor with a network connection to a home work place. The software will have the ability to take information from other sensors to make inferences and corroborate conditions.

Use of the Invention in the Combined Support Center 200

The Combined Support Center 200 is staffed around the clock. Its purpose is to monitor and react to emergent and non-emergent events in the user's home or work place. The staff at the CSC has access to real-time as well as stored information concerning the user, user activities, and the user's environment. Software on the CSC processor 210 helps the staff understand this information.

The CSC staff interfaces with the CSC Processor 210 by using video screens 220. The number of screens and how the information is displayed can be determined by the staff depending on their desires. Below is a discussion of the information and how it is used.

The most important page will be the display of names of incoming callers, their status (such as client, medical professional, caregiver, or relative), and emergencies associated with the call. Calls initiated to a home in response to a detected time critical emergent event (e.g., fall, depression of the help button, or a verbal call for help) will be displayed on the incoming call list. Calls will be prioritized in a first come, first answered basis except calls with associated emergent events will be prioritized over non-emergent event calls.

A critical feature of the invention is the ability to initiate three-way video calls that will display the video image of the two other parties as well as support receiving their audio simultaneously. CSC staff can disconnect but leave the two other parties connected. Use and advantages of three way calls was discussed earlier. CSC staff will have the ability to place callers on hold as well as review previous successful calls and missed calls.

When connected to a caller, the CSC staff will have the ability to select information relative to the caller. For home or work place users, there will be a checklist of information to discuss with the caller to address any time-critical needs. This checklist will be tailored to the person. It will include the location and phone numbers of the user's nearest police, fire, and medical responders. It will then include questions relating to a user's current medical conditions so that the CSC staff will not forget to ask the most critical questions specific to known medical conditions of the caller. The CSC Interface User 220 will also make available to the caller other information such as the current medications and dosing instructions; prescribed diet and exercise regimen; care plans; hospital discharge orders; history of exercise, diet, medication compliance, and physiological symptoms; call history; home layout; maps and directions to the home.

Data for the above as well as data on the user's home or work environment equipment and supporting administrative functions will be stored on CSC data bases. These may or not be co-located at the CSC itself One important activity performed by the CSC Processor 210 is the monitoring of all home or work place systems that it supports. The CSC Processor 210 will monitor the status of users' connectivity, processors, Room Boxes 160, and sensors such as fall detection and Help button devices. When problems occur in a user home or work place 100, the CSC staff will initiate appropriate action to restore the capability. If connectivity to the User's Main Processor 110 is lost, the CSC will initiate actions to restore connectivity. Conversely, users' processors will attempt to find alternate CSCs 200 and PBX Servers 400 to support CSC and users needs. This will ensure uninterrupted monitoring of users in their homes and work places. To ensure that CSCs are ready for this contingency, CSC Data Bases 230 will regularly refresh data on their Users' Main Processors 110, and they will synchronize their current users' data with data stored at back-up locations.

Use of the Invention in Ancillary Locations:

As mentioned, the home and work place users and the CSC staff have the ability to video conference with ancillary parties. These include medical providers such as doctors and nurses; medicine, diet, and exercise coaches; and relatives and friends.

Using this invention, these people will have software hosted on their Ancillary User Processors 310 that will enable them to initiate and receive video calls, and, retrieve and enter information into data bases on the network. The role of the ancillary person relative to the user will determine what information is available and what information can be created on data bases.

### Additional Embodiments and Applications

The above discussion has primarily focused on use of the invention to support home health monitoring and independent living. However, the invention is naturally extensible to use in locations where health monitoring can be conducted such as kiosks or dedicated rooms at public locations such as shopping centers, senior centers, or civic centers to support that portion of the population that may not be able to afford in-home systems and are not within reasonable driving time to 24 hour medical facilities.

Additionally, many of the software applications for monitoring, assessing, or intervening may be easily adapted to support simple devices or complex robotic devices that generally increase the quality of life, assist with living activities (whether they are chores such as cleaning, doing the dishes or laundry, etc) or increasing productivity of the homemaker, the home-office worker, the rancher, or the small business employee.

The invention claimed is:

1. A human care sentry system comprising: computer processors; algorithms to process data and control elements of the system; user interfaces; Room Boxes; environmental sensors; hardware sensors and control devices; physiological sensors; quality of life improvement devices;
assistance devices; data and information storage; remote private branch exchange servers; remote monitoring centers; and remote ancillary user processors and interfaces.

2. A human care sentry system according to claim 1, wherein there is a main computer processor and additional processors as needed to support data processing requirements of the system.

3. A human care sentry system according to claim 1, wherein a computer processor hosts algorithms to perform one or more of the functions selected from the group consisting of: create retrieve, and send data; integrate data from the human care sentry system devices and algorithms; detect and corroborate emergency and potentially harmful events; control devices and the environment; self monitors and automatically switches to back up systems.

4. A human care sentry system according to claim 1, wherein there is a user interface with one or more of the items selected from the group consisting of: video teleconference capability; calendar and appointment reminder; medication compliance monitor; voice generated information; and speech recognition.

5. A human care sentry system according to claim 1, wherein there one or more Room Boxes with one or more of the items selected from the group consisting of; data; control signal; and two way audio relays; a computer processor; movement detection devices; motion detection devices; emergency lights; smoke detection devices; carbon monoxide detection devices; ?.

6. A human care sentry system according to claim 1, wherein there are environmental sensors sensing one or more of the items selected from the group consisting of: room temperature, door position, window position, appliance status (off, on, inoperative).

7. A human care sentry system according to claim 1, wherein there is one or more hardware sensor and control devices selected from the group of the following: quality of life improvement devices, and assistance devices.

8. A human care sentry system according to claim 1, wherein there is one or more physiological data sensing or measuring devices selected from the group of the following: scales to measure body weight, blood oxygen sensor to measure oxygen saturation, blood sugar level measuring device, blood pressure measuring device, and a heart rate measuring device.

9. A human care sentry system according to claim 1, wherein there is a personal fall detector and help request device composed of a computer, three accelerometers mounted orthogonally, a battery, a switch activated by depression, and a wireless transmitter and receiver to pass data from the computer to another computer.

10. A human care sentry system according to claim 1, wherein there is data storage at either or both local and remote locations.

11. A human care sentry system according to claim 1, wherein there are one or more remote monitoring centers with operator interfaces to initiate and engage in multi-party video teleconferences and transfer video calls; computer processors; algorithms to retrieve, create, and send data and information; local data storage; and monitoring of access to data storage and automatic switching to back-up data storage devices.

12. A human care sentry system according to claim 1, wherein there are one or more remote user locations consisting of a computer processor to initiate and engage in multi-party video teleconferences and transfer video calls; computer processors; algorithms to retrieve, create, and send data and information; and local data storage.

13. A software architecture and schema comprising: a common data dictionary; machine readable, self-describing hardware or software component data sheets; network and processor managers; data directories; data bases; Rich Query software modules; and Data Centric Streams software classes.

14. A software architecture and schema according to claim 13 that uses Interface Modules to bridge devices that export or consume data written to the invention schema, to a network operating on a different transport standard.

15. A software architecture and schema according to claim 13 that automatically integrates standards-compliant new devices into the system by an applications' registration process and a component registration process.

16. A software architecture and schema according to claim 13 that automatically reconfigures the system upon the loss of devices from the system by a data lease system and data lease renewal system.

17. A method to assist human living, comprising the steps of: detecting emergencies and addressing them from a remote monitoring location; detecting potentially harmful situations and addressing them either locally or from a remote monitoring location; monitoring and controlling the user's environment, living assistance devices, and utilities; providing reminders to users; and enabling users to video-teleconference with medical professionals, family, friends, and caregivers.

18. The method of claim 17, wherein the emergency events are falls detected by body worn accelerometers or activation of the body-worn help request button; and corroborated by data from motion detectors, verbal inquiries from the user's computer processor, and verbal inquiries from a remote monitoring location.

19. The method of claim 17, wherein the emergency events are addressed by personnel at a remote monitoring location by establishing contact with the invention user and following pre-coordinated, user specific instructions on how to respond to the specific emergency.

20. The method of claim 17, wherein potentially harmful situations are detected by one or more of the following: the use of motion sensors and computer software that is looking for activity to occur in certain times of the day and locations and also looking for lack of activity at certain times of the day and locations; the use of smoke detectors to identify potential fires; the use of carbon monoxide detectors to identify potentially dangerous levels of carbon monoxide; the use of room temperature sensors to identify potentially dangerous temperatures; the use of door and window sensors and computer software to identify a residence that should be secured or a condition that could lead to unsafe room temperatures; the use of physiological data to indicate potentially worsening congestive heart issues, chronic obstructive pulmonary disease, susceptibility to stroke or hypertension, and diabetes; and the use of medications reminder and compliance software to identify potential non-compliance with medicating instructions.

21. The method of claim 17, wherein potentially harmful situations are addressed by the user's computer querying the user about recent activity, and if the response is not satisfactory, the computer informs either the remote monitoring center which initiates verbal queries and follows user specific escalation procedures or a person at an ancillary location who will work with the user to address the issue.

22. The method of claim 17, wherein the user's the environment, living assistance devices, and utilities are monitored by sensing their state and activity, and using computer software to compare that information with what is expected for the user.

23. The method of claim 17, wherein user's the environment, living assistance devices, and utilities are controlled by the computer verbally informing the user of the state of the environment, living assistance devices, and utilities and suggesting an action to return the environment, living assistance devices, and utilities to a normal state, or the computer will send a control signal to control devices to return the environment, living assistance devices, and utilities to a normal state.

24. The method of claim 17, wherein parties at remote or ancillary locations can make entries to the user's calendar, reminder, or medications compliance data base and the user's computer will display or verbally remind the user of events and activities.

25. The method of claim 17, wherein users will be able to use their computer interface to video call to or be called by medical professionals, family, friends, and caregivers.
